(19) United States
(12) Patent Application Publication
(10) Pub. No.: US 2008/0208074 A1
(43) Pub. Date: Aug. 28, 2008

(54) Methods and Systems for Characterizing and Generating a Patient-Specific Seizure Advisory System

(76) Inventors: David Snyder, Bainbridge Island, WA (US); Kent W. Leyde, Sammamish, WA (US)

Correspondence Address: NEUROVISTA / SHAY GLENN, 2755 CAMPUS DRIVE, SUITE 210, SAN MATEO, CA, 94403, US

(21) Appl. No.: 12/035,335

(22) Filed: Feb. 21, 2008

Related U.S. Application Data
(60) Provisional application No. 60/902,580, filed on Feb. 21, 2007.

(51) Int. Cl. A61B 5/0476

(52) U.S. Cl. 600/545

(57) ABSTRACT

A method of developing a brain state advisory system including the following steps: deriving a brain state advisory algorithm; applying the brain state advisory algorithm to patient EEG data to identify occurrences of the target patient brain state (such as, e.g., a pro-ictal state or a contra-ictal state) in the patient EEG data; determining if a performance measure of the advisory algorithm for the target brain state exceeds the performance measure of a chance predictor for the target brain state; and if the performance measure of the advisory algorithm for the target brain state exceeds the performance measure of a chance predictor for the target brain state, storing the advisory algorithm in memory of the brain state advisory system. The invention also includes seizure advisory systems.

The present application claims benefit of U.S. Provisional Patent Application No. 60/902,580, filed Feb. 21, 2007, to Snyder et al., entitled “Methods and Systems for Characterizing and Generating a Patient-Specific Seizure Detection System,” the disclosure of which is incorporated by reference herein in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The present invention relates generally to methods and systems for characterizing and optimizing algorithms and systems. More specifically, the present invention is directed toward patient customized seizure advisory systems and statistical methods and systems for characterizing and generating patient customized seizure advisory algorithms.

One of the most devastating aspects of epilepsy is the uncertainty of when seizures might occur, an uncertainty that transforms brief episodic events into a debilitating chronic condition. For over 30 years, researchers have tried to reduce this uncertainty by identifying electroencephalogram (EEG) signals that would predict the occurrence of a seizure.

The temporal progression of a seizure may be described in terms of intervals: interictal, pro-ictal (including pre-ictal), ictal, and postictal. The interictal interval is comprised of relatively normative EEG. The pro-ictal period represents a state or condition that represents a high susceptibility for seizure; in other words, a seizure can happen at any time. The pro-ictal state or condition wholly encompasses the pre-ictal state, which some researchers classify as the beginning of the ictal or seizure event which begins with a cascade of events. Under this definition, a seizure is imminent and will occur if the patient is in a pre-ictal condition. The EEG characteristics indicative of a pro-ictal interval are not fully understood, but many characteristics have been hypothesized. These include increased spatial synchrony or coherence, localized entrainment of dynamic properties, and changes in EEG amplitude distributions or spectral distributions. If a transition from pro-ictal interval to ictal (seizure) interval occurs, it is in turn followed by a postictal interval characterized by suppression and slowing of the EEG.

While being able to determine that the patient is in a pro-ictal condition is highly desirable, identifying when the patient has entered or is likely to enter a pro-ictal condition is only part of the solution for these patients. An equally important aspect of any seizure advisory system is the ability to inform the patient when they are unlikely to have a seizure for a predetermined period of time (e.g., when the patient has a low susceptibility of seizure or is in a “contra-ictal” state). A more detailed discussion of the identification and indication of a contra-ictal condition may be found in commonly-owned U.S. patent application Ser. No. 12/020,450, filed Jan. 25, 2008, the disclosure of which is incorporated herein by reference.

The effort to develop seizure advisory technology has been hampered by limitations of data recording equipment, inadequate computing power, small/incomplete datasets, and lack of rigorous statistical analysis. With regards to statistical analysis, a majority of published work has suffered from one or more of the following problems:
Lack of statistical power, primarily due to inadequate interictal EEG.
Absence of a statistical control, e.g. chance predictor.
The use of a posteriori information in the assessment of algorithm performance. Specific examples include:
   The use of in-sample data for algorithm testing.
   Retrospective selection of data channels (electrodes) for best performance.
Lack of complete performance characterization: sensitivity, specificity, negative predictive value, positive predictive value.
Inclusion of clustered seizures in sensitivity analysis, despite the lack of statistical independence and intervening interictal condition.

Many of these shortcomings were recently catalogued in a review of more than 40 seizure prediction studies, [Mormann et al. 2006a] in which the authors conclude that “the current literature allows no definite conclusion as to whether seizures are predictable by prospective algorithms.”

### SUMMARY OF THE INVENTION

The failure of earlier seizure prediction algorithms to accurately predict seizures obscures the point that prior algorithms and proposed seizure prediction systems failed to recognize a key point: The question is not whether a seizure is imminent. Rather, the question is whether the patient is in a pro-ictal state, i.e., a state in which the patient is highly susceptible to a seizure, even if the seizure does not ultimately occur before the patient returns to a contra-ictal state or other interictal state. One aspect of the invention therefore provides a reliable seizure advisory system and method that can be used to indicate when a patient is in a pro-ictal state. Such an indication is not a warning that a seizure will necessarily occur but is instead an indication that the patient's current state is one with a heightened susceptibility of a seizure.

Furthermore, while an indication of all occurrences of a pro-ictal state could reliably identify all possible seizures, not every pro-ictal state may result in a seizure. In fact, for reasons that are not well understood, some patients may transition from pro-ictal states to ictal states more often than other patients do, which means that these latter patients would have a higher ratio of time spent in warning to time spent in seizure than the former patients. Therefore another aspect of the invention provides a way to modify the operation of a seizure advisory system to change the time spent in warning for a given set of input EEG conditions.

Of course, a change to a seizure advisory algorithm that reduces time spent in warning could render the algorithm less useful clinically if such change reduces the ability of the algorithm to reliably identify pro-ictal states below a particular threshold. Another aspect of the invention therefore provides a way to determine and indicate the manner in which the algorithm's sensitivity is affected by changes in time spent in warning.

The present invention provides systems and methods for identifying a hypothetical state or condition for a patient in a patient dataset, such as an EEG dataset, that has an unknown and/or variable duration, such as the aforementioned pro-ictal state. The present invention also provides performance metrics that are able to statistically characterize performance characteristics of a system used to identify the hypothetical state or condition. The generated performance metrics may thereafter be used to guide optimization of the system that was used to identify the hypothetical state or condition. In one particular configuration, the systems and methods are directed toward identifying a pro-ictal state for patients that have epilepsy.

The present invention provides systems and methods for optimizing a state advisory system for identification of a hypothetical state known to exist in a point in time having an unknown duration. The method comprises detecting properties of the hypothetical state at the known point in time. The known point in time is approximately at the end of the hypothetical state (e.g., pro-ictal state) and/or at the beginning of the known state (e.g., ictal or seizure state). After the properties of the hypothetical state at the known time are determined, nearby time intervals which have similar state properties as the properties of the hypothetical state at the known point in time are identified. A grouping of adjacent nearby time intervals which have similar state properties as the properties of the hypothetical state at the known point in time are identified as encompassing the hypothetical state. Finally, the identified grouping of nearby time intervals that encompassed the hypothetical state is used to optimize the state detection system.

As it relates to a seizure advisory system, a number of different methods and systems may be used to carry out the above method. For example, the method may move forward in time through a patient dataset toward the onset of the seizure, or it may move backward in time through the patient dataset from the onset of the seizure to identify the nearby time intervals and groupings of time intervals. The time intervals may be sequential and non-overlapping time intervals or the time intervals may be overlapping time intervals. The methods may utilize a block-wise method (e.g., alerts in a prediction window) or a point-wise method (e.g., alerts and coupling intervals)—both of which are described in detail below. The time intervals may be spaced in time from the onset of the seizure, or the time intervals may overlap the onset of the seizure.

Unlike conventional methods, which utilize a time interval that has a fixed duration to identify the pre-ictal state, the present invention has the ability to group time intervals that have similar characteristics so as to provide the capability to fully encompass and characterize pro-ictal periods having an unknown and/or variable duration.

Once the system is able to accurately identify the known state or condition, the statistical methods and metrics described herein may be used to assess the ability of the seizure advisory system to identify the unknown state or condition. The statistical methods and metrics described herein provide consistent definitions that allow for comprehensive characterization of the performance of the system. The metrics include true positive, true negative, false positive, false negative, sensitivity, specificity, negative predictive value, positive predictive value, time in alert, time in false alert, percentage of time in false alert, percentage of time in alert, whether or not the seizure prediction system performs better than a chance predictor, etc. Such metrics are applicable to both the block-wise approach and point-wise approach described herein.

In most embodiments, the positive predictive behavior, such as the sensitivity, specificity, negative predictive value and/or positive predictive value, are characterized to assess the algorithm performance. Once the performance is characterized, some aspect of the system may be modified to improve the performance of the system. Advantageously, such modification will allow for tailoring and optimizing of the system to a particular patient. Some examples of aspects that may be modified include changing features, combining the features with additional features, changing classifiers, moving a threshold or other decision criteria of an existing classifier, changing a shape of a threshold or other decision criteria of the existing classifier, etc.

The systems and methods of the present invention further enable the testing and development of a practical implementation of a seizure advisory system. In one configuration, the system matches a false positive behavior of the seizure advisory system to the needs of a patient. For example, in one embodiment, the seizure rate of the patient is matched to some multiple (e.g., 1:1, 1.5:1, 2:1, etc.) of the false positive rate of a seizure advisory system. Thus, if the patient has one seizure a week, the seizure advisory system is allowed to only have one false positive a week. Of course, the present invention is not limited to the use of false positive rate or the seizure rate, and other characteristics may be used to characterize performance of the seizure prediction system. Some examples of other characteristics include the number of false positive for a time interval, time in false positive for a time interval, percentage of time in false positive for a time interval, or the like.

After the false positive prediction behavior is substantially correlated to the needs of the patient, a true positive identification behavior of the seizure advisory system is measured. Depending on the results of the measurement, some parameter of the seizure advisory system may be modified so as to improve the performance of the seizure advisory algorithm for the particular patient.

Based on the above methods and systems, the present invention provides a complete tool set that enables generation of a patient-tailored prediction system that has comprehensive performance characteristics measured.

In one embodiment, the present invention provides a seizure advisory system that performs better than a chance predictor. The seizure advisory system comprises a sensitivity of greater than 70% when a false positive (FP) rate is substantially matched to a patient's seizure rate. In particular implementations, the seizure advisory system comprises a sensitivity greater than 75%, greater than 90%, and greater than 94%.

One aspect of the invention provides a method of developing a brain state advisory system including the following steps: deriving a brain state advisory algorithm; applying the brain state advisory algorithm to patient EEG data to identify occurrences of the target patient brain state (such as, e.g., a pro-ictal state or a contra-ictal state) in the patient EEG data; determining if a performance measure of the advisory algorithm for the target brain state exceeds the performance measure of a chance predictor for the target brain state; and if the performance measure of the advisory algorithm for the target brain state exceeds the performance measure of a chance predictor for the target brain state, storing the advisory algorithm in memory of the brain state advisory system. The method may also include the step of generating an alert when the target brain state is identified.

In some embodiments, the performance measure is a first performance measure, and the method further includes the step of determining an operating point of the chance predictor at which a second performance measure of the chance predictor is substantially the same as the second performance measure of the advisory algorithm prior to determining if the first performance measure of the advisory algorithm exceeds the first performance measure of the chance predictor. The first and second performance measures may be complementary performance measures, such as sensitivity and specificity; sensitivity and percent time in alert; and/or negative predictive value and percent time in contra-ictal indication.

Another aspect of the invention provides a method of monitoring a patient brain state including the following steps: obtaining EEG data from the patient; analyzing the EEG data with a stored brain state advisory algorithm having a performance measure for identification of a target brain state (such as, e.g., a pro-ictal state or a contra-ictal state) exceeding the performance measure of a chance predictor for the target brain state; and providing an indication of the target brain state.

In some embodiments, the performance measure is a first performance measure, the analyzing step including the step of analyzing the EEG data with a stored brain state advisory algorithm having a first performance measure for identification of a target brain state exceeding the first performance measure of a chance predictor for the target brain state, wherein a second performance measure of the chance predictor for identification of the target brain state is substantially equal to the second performance measure of the stored advisory algorithm for identification of the target brain state. Once again, the first and second performance measures may be complementary performance measures, such as sensitivity and specificity; sensitivity and percent time in alert; and/or negative predictive value and percent time in contra-ictal indication.

Still another aspect of the invention provides a seizure advisory system having a seizure advisory algorithm stored in memory, the seizure advisory algorithm having a performance measure for identifying a target brain state (such as, e.g., a pro-ictal state or a contra-ictal state) greater than the performance measure of a chance predictor for the target brain state; patient EEG data input; a microprocessor programmed to apply the algorithm to EEG data from the patient EEG data input to compute patient brain state; and a patient brain state indicator controlled by the microprocessor to indicate patient brain state.

In some embodiments, the performance measure is a first performance measure, the seizure advisory algorithm having a first performance measure for identifying the target brain state greater than the first performance measure of a chance predictor for the target brain state, the seizure advisory algorithm having a second performance measure for identifying the target brain state that is substantially equal to the second performance measure of the chance predictor for the target brain state. The first and second performance measures may be complementary performance measures, such as sensitivity and specificity; sensitivity and percent time in alert; and/or negative predictive value and percent time in contra-ictal indication.

Yet another aspect of the invention provides a method of developing a brain state advisory system including the following steps: deriving a brain state advisory algorithm, the deriving step including analyzing patient EEG data (such as, e.g., patient EEG data that preceded a seizure by more than 90 minutes), identifying all pro-ictal states within the EEG data, and generating pro-ictal state alerts; and placing the advisory algorithm in memory of the brain state advisory system. In some embodiments, the step of identifying all pro-ictal states includes the step of identifying all pro-ictal states within the patient EEG data without regard to time prior to seizure.

In some embodiments, the deriving step further includes the step of adjusting sensitivity of the algorithm in identifying pro-ictal states, such as by modifying a ratio of number of pro-ictal state alerts generated in the generating step to number of seizures in the EEG data; modifying a percentage of time encompassed by pro-ictal alerts generated in the generating step; and/or modifying a percentage of time encompassed by pro-ictal alerts generated in the generating step that do not terminate in a seizure. In some embodiments, the step of identifying all pro-ictal states includes the step of treating a clustered seizure as a single event.

In some embodiments, the step of generating all pro-ictal state alerts includes the step of maintaining a pro-ictal alert for a predetermined periodic of time after entering a pro-ictal state, possibly even after ceasing to identify a pro-ictal state in the EEG data. The pro-ictal state alert may be extended for a second predetermined period of time if a pro-ictal state is again identified after the ceasing step and before the first predetermined period of time has expired.

Still another aspect of the invention provides a method of monitoring a patient brain state including the following steps: obtaining EEG data from the patient; analyzing the EEG data with a stored brain state advisory algorithm; and providing an indication of a pro-ictal brain state for a predetermined period of time after identification of the pro-ictal brain state. In some embodiments, the providing step includes the step of continuing the indication of a pro-ictal brain state after the algorithm has ceased to identify a pro-ictal brain state. Once again, the pro-ictal state alert may be extended for a second predetermined period of time if a pro-ictal state is again identified after the ceasing step and before the first predetermined period of time has expired.

Another aspect of the invention provides a seizure advisory system having a seizure advisory algorithm stored in memory; patient EEG data input; a microprocessor programmed to apply the algorithm to EEG data from the patient EEG data input to identify and indicate patient brain state; and a patient brain state indicator controlled by the microprocessor to indicate patient brain state for a predetermined period of time after identification of a pro-ictal brain state. In some embodiments, the microprocessor is programmed to control the patient brain state indicator to indicate patient brain state for a predetermined period of time after identification of a pro-ictal brain state even if the algorithm has ceased to identify a pro-ictal brain state. The microprocessor may also be programmed to control the patient brain state indicator to extend an indication of a pro-ictal brain state for a second pre-determined period of time if the algorithm identifies another pro-ictal brain state before the first predetermined period of time has expired.

Yet another aspect of the invention provides a method of developing a brain state advisory system including the steps of: deriving a brain state advisory algorithm, the deriving step including analyzing patient EEG data, identifying pro-ictal states within the EEG data, and generating pro-ictal state alerts; adjusting a pro-ictal state identification sensitivity of the algorithm; and storing the advisory algorithm in memory of the brain state advisory system. In some embodiments, the adjusting step may be performed by modifying the identifying step and/or modifying the generating step.

In some embodiments, the adjusting step includes the step of reducing a ratio of number of pro-ictal state alerts generated in the generating step to number of seizures in the EEG data; modifying a percentage of time encompassed by pro-ictal alerts generated in the generating step; and/or modifying a percentage of time encompassed by pro-ictal alerts generated in the generating step that do not terminate in a seizure. In some embodiments, the generating step includes the step of generating alerts each having an alert duration and wherein the adjusting step comprises adjusting a ratio of cumulative alert durations to total time of the EEG data.

Still another aspect of the invention provides a method of tailoring a seizure advisory system to a patient including the following steps: correlating a first performance measure of the seizure advisory algorithm to a seizure behavior of a subject (such as, e.g., a number of seizures in a time interval); modifying an aspect of the seizure advisory algorithm to improve a second performance measure of the seizure prediction system (such as to, e.g., tailor the seizure advisory system to a particular patient); and storing the algorithm in memory in the seizure advisory system. The first and second performance measures may be complementary performance measures, such as sensitivity and specificity; sensitivity and percent time in alert; and/or negative predictive value and percent time in contra-ictal indication. In some embodiments, the seizure advisory algorithm includes a feature extractor and a classifier.

In some embodiments, the step of modifying an aspect of the seizure advisory algorithm includes the step of modifying a feature vector analyzed by the seizure advisory algorithm; changing feature extractors or combining the feature extractor with an additional feature extractor; and/or moving or changing a shape of a boundary between classes identified by the classifier.

Yet another aspect of the invention provides a method of improving performance of a seizure advisory system, the seizure advisory system comprising a seizure advisory algorithm, the method including the steps of: applying the seizure advisory algorithm to a dataset to generate alerts; extracting information related to alert duration during a time interval of the dataset; modifying at least one parameter of the seizure advisory algorithm to improve performance of the seizure advisory system; and placing the seizure advisory algorithm in memory of the seizure advisory system.

Another aspect of the invention provides a method for optimizing a state detection algorithm for detection of a hypothetical state having a known conclusion and unknown onset, the method including the following steps: identifying the known conclusion of the hypothetical state; analyzing a first time interval to determine if the time interval is similar to the hypothetical state; analyzing one or more time intervals forward in time from the first interval to determine if the sequential intervals are suitably similar with the hypothetical state; determining if the first time interval and one or more sequential intervals overlap with each other and if at least one of the sequential intervals overlaps with the known conclusion of the hypothetical state, and if so, then defining the first interval and the sequential interval(s) as being within the known conclusion and unknown onset; identifying a grouping of sequential time intervals which are similar to the hypothetical state; using the identified grouping of sequential time intervals to optimize the state detection algorithm; and storing the state detection algorithm in a seizure advisory system.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a block diagram illustrating aspects of feature extractors and classifiers.

FIG. 2A illustrates a prior art alert (arrow) that is a true positive (TP).

FIG. 2B illustrates a prior art alert (arrow) that is a false positive (FP).

FIG. 3 illustrates algorithm outputs for three different threshold levels.

FIG. 4 illustrates the process of defining detection and prediction windows, alert and non-alert windows and the evaluation of true negative (TN), true positive (TP), false positive (FP) and false negative (FN) prediction windows.

FIG. 5A illustrates an example of a true negative (TN), true positive (TP) and an alert duration.

FIG. 5B illustrates an example of an extended TP and an alert duration.

FIG. 5C illustrates an example of a false positive (FP) and a false negative (FN).

FIG. 6 illustrates examples of TN, FP, TP for an embodiment that includes coupling intervals with each alert (arrow).

FIG. 7A illustrates a seizure epoch.

FIG. 7B illustrates an interictal epoch.

FIG. 8A illustrates a continuous EEG record that includes interictal epochs, ictal epochs, “other epochs” used for training, and “other epochs” not used for training.

FIG. 8B illustrates a hold-out validation method.

FIG. 8C illustrates a second fold of a 2-fold cross-validation method without holdout (FIG. 8B) as the first fold).

FIG. 8D illustrates a randomized 2-fold cross validation.

FIG. 9 is a schematic representation of a leave-one-out cross-validation with extra folds for testing of “other epochs.”

FIG. 10 illustrates nine traces that represent feature values calculated from a three-by-three section of a subdural electrode grid located over the origin of seizure activity.

FIG. 11 illustrates classifier outputs derived from the feature calculations illustrated in FIG. 10.

FIG. 12 illustrates alert signals for three different patients over the course of their EMU stay wherein the long vertical bars indicate a seizure, the short unmarked vertical bars are TP and the marked vertical bars are FP.

FIG. 13 illustrates sensitivity distribution when false positives are matched to the number of seizures.

FIG. 14 illustrates alert durations when false positives are matched to the number of seizures, wherein the upper portion illustrates 0 to 1000 minutes and the lower portion is an enlarged view from 0 to 100 minutes.

FIG. 15 illustrates sensitivity distributions when 2 false positives are allowed for each seizure.

FIG. 16 illustrates alert durations when 2 false positives are allowed for each seizure. The upper portion of FIG. 16 illustrates a large time scale and the lower portion illustrates an enlarged view from 0 to 100 minutes.

FIG. 17 illustrates a distribution of sensitivity for 57 patients with two-fold cross validation, and across 10 different epoch randomizations of leave-one-out cross-validation. Box plots indicate population median, quartiles, and ten percentiles.

FIG. 18 is one embodiment of a simplified seizure advisory system which has an array of epidural or subdural electrodes and an array of depth electrodes in communication with an external assembly through an implanted assembly;

FIG. 19 is a block diagram of an implanted communication unit that may be used in accordance with the systems and methods described herein;

FIG. 20 is a block diagram of an external data device that may be used in accordance with the systems and methods described herein;

FIG. 21 is an external assembly that may be used with the seizure advisory system of this invention;

FIG. 22 is a user interface including outputs of an exemplary external assembly that may be used with the seizure advisory system of this invention;

FIG. 23 is an example timeline for a typical therapeutic regimen for the treatment of epilepsy; and

FIG. 24 is an example timeline for a therapeutic regimen for the treatment of epilepsy that may be enabled by the system and methods described herein.

FIG. 25 illustrates an example algorithm performance report for the patient and/or physician to assist in tailoring the algorithm to the patient.

### DETAILED DESCRIPTION OF THE INVENTION

While the remaining discussion focuses on monitoring brain activity for detecting and/or determining the susceptibility of an onset of a seizure, the present invention may be equally applicable to monitoring and treatment of other neurological and non-neurological conditions. For example, some other conditions that may be treated using the systems of the present invention include, but is not limited to, Alzheimer' disease, Parkinson's disease, migraine headaches, sleep apnea, Huntington's disease, hemiballism, choreoathetosis, dystonia, akinesia, bradykinesia, restless legs syndrome, other movement disorder, dementia, depression, mania, bipolar disorder, other affective disorder, motility disorders, anxiety disorder, phobia disorder, borderline personality disorder, schizophrenia, multiple personality disorder, and other psychiatric disorder, Parkinsonism, rigidity, or hyperkinesias, addiction, substance abuse, attention deficit hyperactivity disorder, impaired control of aggression, impaired control of sexual behavior, or the like.

Furthermore, while preferred embodiments of the present invention analyze EEG recordings (extracranial EEG, intracranial EEG, etc.), the methods of the present invention may also be applicable to other physiological signals or changes in the other physiological signals, such as magnetoencephalography, blood pressure, oxygen availability, blood oxygenation indicator via pulse oximetry, temperature of the brain or of portions of the patient, blood flow measurements, ECG/EKG, heart rate signals, respiratory signals, chemical concentrations of neurotransmitters, chemical concentrations of medications, pH in the blood, other vital signs, other physiological or biochemical parameters of the patient's body, or the like.

### Seizure Advisory System

The development of a seizure advisory system according to this invention typically involves the following steps: Development of a general seizure advisory algorithm; deployment of the general algorithm in a patient seizure advisory device; and adapting the general seizure advisory algorithm to a particular patient. The general algorithm may be based on data (e.g., EEG data) from more than one patient, or it may be based on data from only one patient. In some embodiments, the adapting step may be performed before deploying the algorithm in the seizure advisory device.

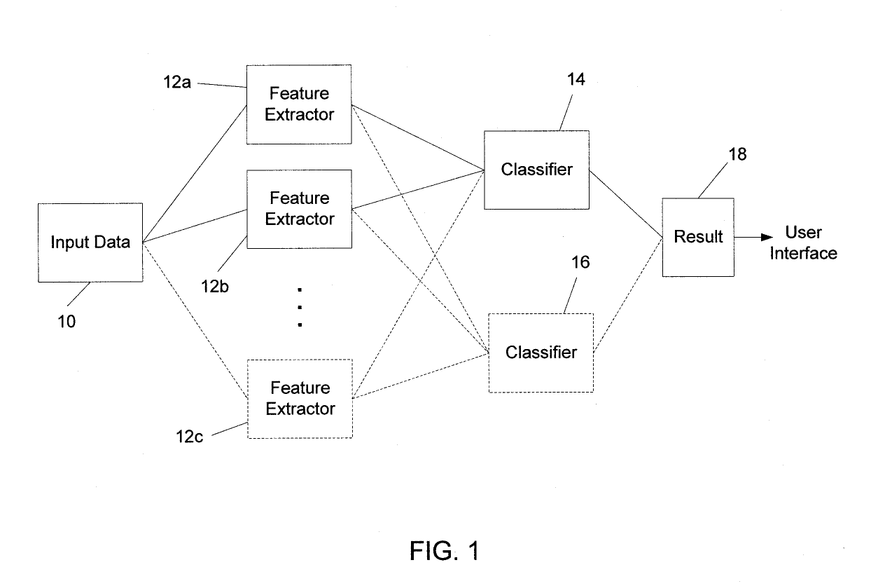

The process of determining a patient's susceptibility to seizures typically comprises four or more algorithm steps as illustrated in a simplified form in FIG. 1. The first step comprises measuring a sequence of biological signals 10 believed to contain information about a patient's propensity for seizure, e.g., the patient's EEG.

The second step quantifies the biological signals (typically with feature extractors 12a, 12b, 12c), in order to capture a desired feature of the data. Such features include univariate features (operating on a single input data channel), bivariate features (operating on two data channels), and multivariate features (operating on multiple data channels). Some examples of potentially useful features to extract from signals for use in determining the subject's susceptibility for a neurological event, include but are not limited to, bandwidth limited power (alpha band [8-13 Hz], beta band [13-18 Hz], delta band [0.1-4 Hz], theta band [4-8 Hz], low beta band [12-15 Hz], mid-beta band [15-18 Hz], high beta band [18-30 Hz], gamma band [30-48 Hz], high frequency power [>48 Hz], bands with octave or half-octave spacings, wavelets, etc.), second, third and fourth (and higher) statistical moments of the EEG amplitudes or other features, spectral edge frequency, decorrelation time, Hjorth mobility (HM), Hjorth complexity (HC), the largest Lyapunov exponent L(max), effective correlation dimension, local flow, entropy, loss of recurrence LR as a measure of non-stationarity, mean phase coherence, conditional probability, brain dynamics (synchronization or desynchronization of neural activity, STLmax, T-index, angular frequency, and entropy), line length calculations, first, second and higher derivatives of amplitude or other features, integrals, and mathematical linear and non-linear operations including but not limited to addition, subtraction, division, multiplication and logarithmic operations. Of course, for other neurological conditions, additional or alternative characteristic extractors may be used with the systems described herein.

Thirdly, the feature values are gathered together and analyzed in order to forecast a likelihood or susceptibility of being in a pro-ictal state. This step typically uses one or more classifiers 14, 16 and is referred to as “classification,” since the result 18 is often expressed as a likelihood of membership in one of the known “classes”: contra-ictal, interictal (between seizures), pro-ictal (high susceptibility state), ictal (seizure), or postictal (post seizure state). For example, a classifier output indicating a high probability of belonging to the pro-ictal class could serve as an alert signal.

The fourth step is employed to process the classifier outputs and produce a desirable user interface. For example, a momentary pro-ictal classification could be used to turn on a red warning light for a period of 15 minutes. If another alert occurs within the 15 minute period, the red light is extended for another 15 minutes. Such a process has the effect of translating intermittent pro-ictal activity into a continuous red warning light—a much more acceptable means of communication from a patient perspective than frequent intermittent red-light warnings.

The following discussion of the algorithm focuses on classifier outputs and applying statistical tests to determine whether or not the EEG contains information that is indicative of a pro-ictal state. Additional metrics are described to provide estimates of performance relevant to different brain states and user interface realizations, but are not utilized for hypothesis testing. Potential embodiments of the user interface are also described.

### Continuous Data

Seizure prediction studies have typically relied on selected non-continuous EEG recordings from a handful of patients, with a minimal amount of interictal data (one to a few hours per seizure). This approach appears to rely on an assumption that the only EEG data relevant to a seizure prediction is the data that preceded an actual seizure by a short period of time, the period of time many researchers would call pre-ictal. In fact, however, there may be a great deal of relevant information in all EEG data between seizures, such as, e.g., EEG data preceding a seizure by six hours. For example, the patient may have experienced pro-ictal brain states that did not immediately (or ever) result in a seizure. Information about these brain states is important in developing a seizure advisory system. The patient may have also experience contra-ictal brain states during that time. Information about these brain states may be useful in developing an advisory algorithm as well.

The paucity of interictal data used as the basis of prior seizure prediction algorithms makes characterization of the percentage of time in warning, the percentage of time in false warning, the false warning rate, specificity, and negative predictive value highly problematic for those algorithms. Furthermore, clustered seizures were often utilized to calculate the sensitivity statistics of prior algorithms even though they are statistically dependent events. It is often the case in such studies to find that the algorithm fails to anticipate the first (primary) seizure in a cluster while correctly “predicting” the subsequent seizures. It is debatable whether such performance is truly seizure prediction, or merely detection of post-ictal suppression and slowing—an eventuality that unrealistically inflates the calculated sensitivity of the algorithm.

To address these shortcomings, the systems and methods described herein utilized a prospective multi-center data collection effort in which continuous intracranial EEG recordings were obtained from patients undergoing evaluation in epilepsy monitoring units (EMU) and archival recordings of continuous EEG were obtained from multiple sources worldwide. The only inclusion criterion was that enough data be available for cross-validation: a minimum of 2 well-isolated electrographic seizures and at least 6 hours of interictal data. Of course, other inclusion criteria could be used, if desired.

A database used to develop the methods and systems described herein is summarized in TABLE 1.

TABLE 1
EEG Database Summary, February 2007

| | |
|---|---|
| Subjects | 57 |
| Age | 11-58 years |

Electrodes

| | |
|---|---|
| Subdural only | 22 |
| Depth only | 13 |
| Both | 22 |
| Primary Seizures (Clustered Seizures Excluded) | 230 |

Duration of EEG recordings

| | |
|---|---|
| Interictal | 3825 hours |
| Total | 4368 hours |
| Ratio of Interictal Data to Number of Seizures | 17 hours/seizure |

### Full Characterization

The need for comprehensive characterization in the present invention arises from the prior practice of focusing solely on prediction sensitivity (percentage of seizures anticipated), without examination of complementary performance measures, such as percentage of time spent in warning. As a result of this practice, very high sensitivities have been reported, e.g., greater than 80%, which under retrospective analysis have been shown no better than what might be achieved by chance. To illustrate this shortcoming and to illustrate the idea of complementary performance measures, consider the following algorithm:
Mark 10 slips of paper with the integers 1 through 10 and place them in a hat.
Every hour on the hour, randomly draw a slip of paper from the hat, note the number, and return it to the hat.
If the number is 8 or less, predict a seizure within the next hour, otherwise predict no seizure

For any given seizure, there is an 80% probability that the previous random drawing resulted in a correct prediction, yielding a sensitivity of 80%. The weakness of this algorithm is apparent, however, when specificity and percentage of time in warning are examined. For any given hour without seizure, there is only a 20% probability that the previous random drawing resulted in a correct prediction, yielding a wholly unsatisfactory specificity of 20% and leaving the patient in a state of alert 80% of the time. Under these scoring rules, sensitivity and specificity are defined as complementary measures, since for a chance predictor one can be improved only at the expense of the other. For example if in the above experiment a seizure is predicted if the number is 9 or less, the sensitivity is improved to 90%, but the specificity is reduced to 10%. Sensitivity and percentage of time in warning are similarly complementary, since improving sensitivity to 90% undesirably increases the time in alert to 90%.

Sensitivity (Sn), specificity (Sp), negative predictive value (NPV), and positive predictive value (PPV) are typically defined in terms of the number of true and false positive predictions (TP and FP), and true and false negative predictions (TN and FP):

$$\begin{matrix}{S}_{n} = \frac{T P}{T P + F N} & ( 1 a ) \\ {S}_{p} = \frac{T N}{T N + F P} & ( 1 b ) \\ N P V = \frac{T N}{T N + F N} & ( 1 c ) \\ P P V = \frac{T P}{T P + F P} & ( 1 d )\end{matrix}$$

It is important to note that sensitivity is an appropriate metric for evaluation of seizure prediction algorithms only if the patient is unable to alter the course of an impending seizure based on information provided by the algorithm. This condition is met, of course, when an algorithm is applied to prerecorded data. It can even be true in a prospective clinical trial as long as the patient is blinded to pro-ictal warnings.

Since sensitivity can be scored only against seizures that occur, and not against those that are prevented, it has little utility once a patient is provided with pro-ictal warnings. If all impending seizures are prevented by effective treatment, e.g. responsive drug therapy, a highly effective seizure advisory algorithm would be rewarded with a sensitivity score of zero. Thus a best-case treatment receives a worst-case score. A more appropriate metric under this scenario might be the rate of unanticipated seizures.

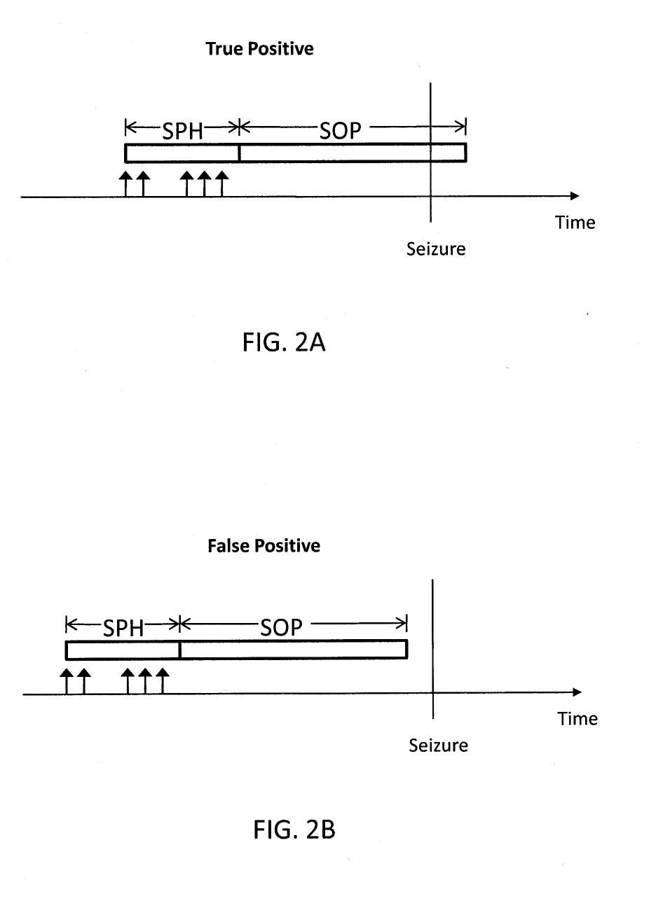

The simplicity of these equations is deceptive, since precise definition of TP, FP, TN, and FN in prior algorithms has proven to be problematic. To appreciate the difficulty, consider a recent study that defined TP and FP with respect to the characteristics of a desired intervention [Winterhalder et al. 2003]. As shown in FIGS. 2A and 2B, the seizure prediction horizon (SPH, also referred to as intervention time, IT [Schelter et al. 2006]) is defined as the minimum time required for a desired intervention to become effective. The seizure occurrence period (SOP) represents the window during which the seizure is expected to occur as determined by the uncertainty inherent in the seizure prediction algorithm (FIGS. 2A and 2B). An alert (indicated by vertical arrows) is counted as a TP if a seizure occurs within the SOP (FIG. 2A), and FP otherwise (FIG. 2B). A user interface for an algorithm consistent with these definitions, for example, might turn on a warning light at the moment of the first alert, and hold it on for the duration SPH+SOP.

This proposal has the virtue of recognizing the temporal uncertainty of seizure prediction, but has several undesirable properties:
Calculated algorithm performance is intervention centric rather than human centric, i.e., it answers the question “is the algorithm suitable for a particular intervention” rather than “what are the characteristics of the algorithm and what interventions might therefore be suitable.”
The notion of uncertainty in the duration of a seizure warning is captured in the duration of the SOP, which nonetheless depends on rigid time periods. This begs the question of whether an alert should be counted as false if a seizure occurs 1 second after the end of the SOP? 5 seconds? 1 minute?
Specificity cannot be calculated, since no corresponding definitions for TN and FN are offered. The rate of false alerts is proposed as an alternative, but no mechanism is provided for calculating the percentage of time spent in false alert.

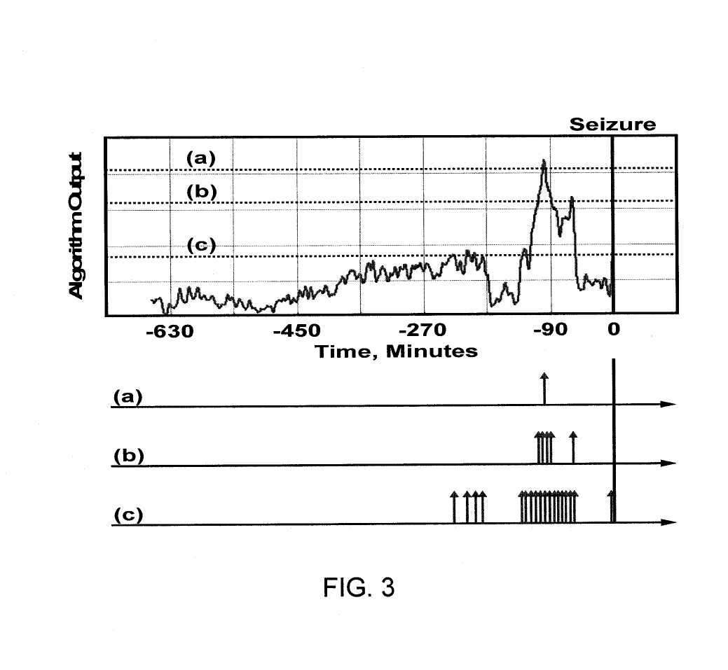

Another complication is presented by the manner in which alerts are issued by a seizure prediction algorithm. A typical technique in prior seizure prediction systems is to raise an alert whenever an algorithm output exceeds a threshold value. FIG. 3 shows three different threshold values (a), (b) and (c). It is evident from FIG. 3 that legitimate alerts may be intermittent rather than continuous in nature. Furthermore, what appears to be a momentary false alert may be revealed by adjustment of the threshold to be part of a single long-duration neurological event—an event that should be counted as a true positive.

Finally, the definition of false positive assumes that an indication or warning that the patient is in a pro-ictal state was “false” if no seizure results at all. In fact, however, it is possible that in this false warning the algorithm and system accurately identified and warned of a state in which a seizure was just as likely as when a prior or subsequent warning did result in a seizure. The warning therefore is not “false.” It accurately identified the patient's state, even if the patient did not have a seizure.

To address these shortcomings the embodiments of the present invention provide one or more (and preferably all of) the following properties that are desirable for a seizure advisory metric:
Consistent calculations of complementary performance characteristics.
A mechanism for calculating the percentage of time spent in alert and/or false alert.
The ability to recognize clustered intermittent alerts as a single neurological event.
Characterize alert duration in a continuous manner, rather than requiring alerts to fall within rigid windows.

The above seizure advisory metrics allow for complete characterization and assessment of the performance of the seizure advisory system. Such metrics will further allow for modification of one or more aspects of the seizure advisory system so as to improve the system performance for the population or a particular patient.

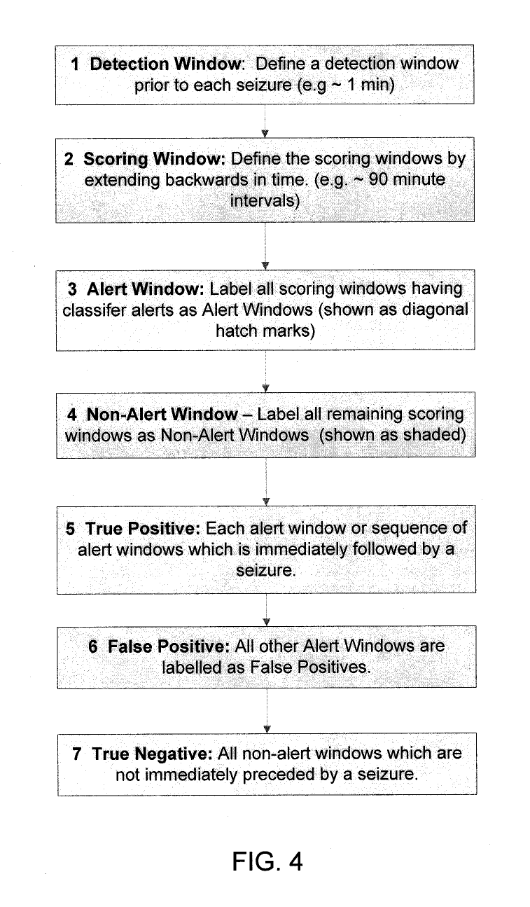

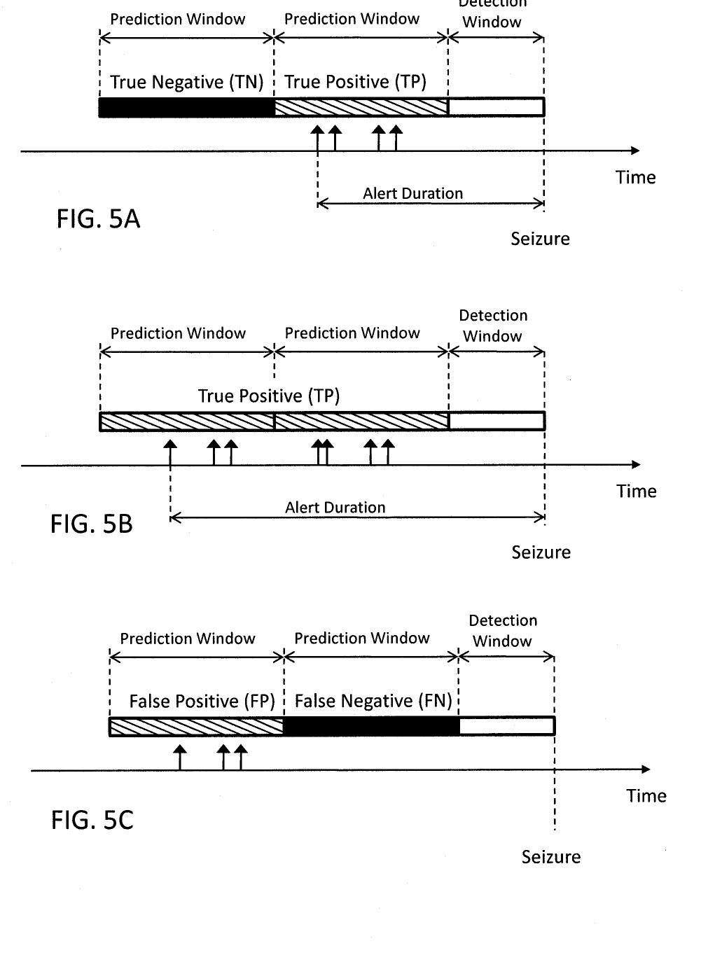

In one embodiment, the methods and systems of the present invention use a block-wise approach for defining TP, FP, TN, and FN, based on “scoring windows” and “detection windows” (FIGS. 5A-C). The method is designed to test, in a relatively simple manner, whether classifier outputs preceding seizures are statistically different from those observed during interictal intervals, such as classifier outputs corresponding to contra-ictal brain states. This information is used to identify classifier outputs corresponding to pro-ictal brain states. The method as outlined in FIG. 4 also provides information on alert duration, and an approximation of the percentage of time spent in alerts that did not result in a seizure, without regard to user interface implementation.

In Step 1 of FIG. 4 a detection window is first defined prior to each seizure, as shown in FIG. 5A. Its purpose is to exclude seizure detection from the statistics of seizure prediction. Classifier outputs from the detection window are therefore not used in developing pro-ictal classifications for the advisory algorithm. The detection window should be large enough to encompass any uncertainty as to seizure onset time that might be introduced during an EEG review and annotation process—1 minute generally suffices, but it could be a longer or shorter period, as desired.

In Step 2 of FIG. 4 “scoring windows” are defined extending backwards in time from the detection window. In Step 3 scoring windows containing one or more classifier alerts (i.e., extracted features classified as pro-ictal) are labeled as “alert windows” (shown as diagonal hatch marks). Scoring windows with a complete absence of classifier alerts are labeled as “non-alert windows” (shown as shaded). FIG. 5A illustrates two scoring windows. The one nearest the seizure has four classifier alerts. It is considered an alert window and is therefore marked with diagonal hatch marks. The scoring window prior is considered a non-alert window as it contains no classifier alerts and is therefore shaded. In Step 5 true positive alert windows are determined. A true positive is counted for each alert window that immediately precedes a seizure, as shown in FIG. 5A.

True positive pro-ictal intervals longer than a scoring window are accommodated as shown in FIG. 5B, wherein sequential alert windows are counted as a single true positive as long as the final scoring window in the sequence immediately precedes the seizure. This remains consistent with Equation 1a and the definition of sensitivity as the percentage of seizures that are correctly identified. In Step 6 false positive alert windows are determined. All other alert windows which are not true positive are counted as false positives as shown in FIG. 5C, recognizing, of course, that these false positives may in fact be indicative of a pro-ictal state and may be useful in a seizure advisory system, as mentioned above. Unlike true positives, sequential false positive scoring windows are counted individually, and are not consolidated into a single FP count. This is done to provide the most conservative estimate of specificity according to equation 1b. Importantly, sensitivity and specificity are no longer complementary performance measurements, since both sensitivity and specificity of a chance predictor can be improved simultaneously, as might be appreciated from FIG. 5 where a TN in FIG. 5A becomes part of a TP in FIG. 5B. “Perfect” performance can be achieved by simply creating a permanent alert—all seizures are anticipated and specificity is perfect since there are no FP—a clearly undesirable result.

In Step 7 true negative windows are determined. True negatives are counted for each non-alert window that does not immediately precede a seizure (as shown in FIG. 5A), otherwise a false negative is counted (as shown in FIG. 5C). The alert duration is characterized by the time between the first classifier alert generating a TP and seizure onset (FIGS. 5A and 5B). The alert duration is thus a continuous metric, independent on the block-wise statistical model.

With these definitions, it is possible to approximate the percentage of time spent in alert and/or false alert by Equation 2 where FP represents the total time spent in scoring windows determined to be false positive divided by the total amount of time (Time in Alert and Non-Alert Windows):

$$\begin{matrix}Alert Percentage ≅ \frac{( AlertWindows )}{( Alert Windows ) + ( Non \text{-} Alert Windows )} & ( 2 a ) \\ False Alert Percentage ≅ \frac{F P}{( Alert Windows ) + ( Non \text{-} Alert Windows )} & ( 2 b )\end{matrix}$$

Equations 2 are approximate for the following reasons. If two classifier alerts are issued just a few seconds apart, yet span the boundary between two scoring windows, they will be counted as two windows of time—an overly conservative view. On the other hand, user interface implementations may extend the time in false alert beyond what may be estimated by Equation 2. For example, a user interface that turns a red light on for 90 minutes following a classifier alert may extend the time in false alert beyond the window in which the alert occurred. Additionally, the denominators in Equations 2 does not take into account the detection window, the duration of a seizure or the time spent in post-ictal condition.

The described methodology is able to calculate sensitivity, specificity, percentage of time spent in alert and percentage of time spent in false alert. It also offers several additional strengths:
Performance metrics are human centric rather than intervention centric, i.e., they do not depend on the characteristics of any particular intervention.
Performance metrics are independent of any specific user interface implementation.
Alert durations are calculated in a continuous manner, independent of the block-wise statistical model.
Seizure detection is differentiated from seizure prediction and pro-ictal state identification, and a mechanism is provided to accommodate uncertainty in annotation of the precise time of seizure onset.

In order to identify an appropriate duration for the scoring window, consider the algorithm output and alert patterns illustrated in FIG. 3. A very strong pro-ictal signal begins approximately 120 minutes prior to the seizure (as indicated by high algorithm output in FIG. 3), continues for approximately 70 minutes, and is followed by 50 minutes of relatively normal EEG leading into a seizure. This sequence of pro-ictal activity followed by a period of suppression prior to seizure is a common feature of many patients in the database. If the scoring window is made too short (less than 50 minutes), the pro-ictal behavior is counted as a false positive event. On the other hand, a scoring window that is too large (e.g., hours) greatly reduces the number of windows available during a patient's interictal periods. This can lead to the reporting of low false prediction rates that nonetheless represent a large proportion of interictal time spent in a state of false alert [Mormann et al. 2006b]. Our experience has shown that a scoring window of 90 minutes is sufficient to capture most such phenomena (though not all) in the existing database. As such, the scoring window could be longer or shorter as desired, and the scoring window may be customized for each particular patient, depending on their particular situation.

With these definitions in place, equations 1a-b and 2 provide a set of metrics that are able to completely characterize the performance of a seizure advisory algorithm.

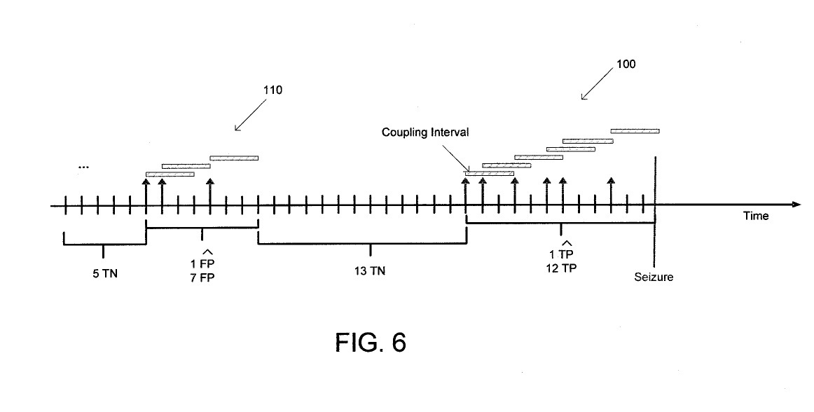

In another embodiment, the present invention provides methods and systems that employ a point-wise approach for defining TP, FP, TN, and FN, based on “coupling intervals” (FIG. 6). Similar to the embodiments of FIGS. 5A-5C, this embodiment is also designed to test whether classifier outputs preceding seizures are statistically different from those observed during interictal intervals. The method also provides information on alert duration and an approximation of the percentage of time spent in false alert that is independent of user interface implementation.

According to this approach, the method moves forward in time from sampling period to sampling period to characterize the performance of a seizure prediction algorithm. Since the point-wise approach is able to scale the “scoring windows” down to the classifier sampling rate, this point-wise approach provides improved time resolution and substantially eliminates discontinuities that may arise from the block-wise methods described above when the block size is changed slightly. The classification results of the illustrated embodiment of FIG. 6 are produced at intervals of 1 second.

The method of FIG. 6 further has the advantage of being directly implementable as a user interface by turning on or continuing a warning light during each of the illustrated coupling intervals. In this case, the information on alert duration and percentage of time spent in false alert are exact, rather than approximate.

According to this approach, each classifier alert (shown as an arrow in FIG. 6) generates a “coupling interval” (shown as a horizontal bar) that extends forward in time from the classifier alert for a desired time period. The coupling interval may extend over any time interval, but typically extends between 2 seconds and 240 minutes, and preferably between about 10 minutes and about 240 minutes. The use of a coupling interval provides the ability to characterize an alert duration in a continuous manner and is able to accommodate the intermittent nature of the patient's neurological condition and the unknown duration of the pro-ictal period. If a second alert occurs during the coupling interval of the alert, the two alerts are considered to be part of a “chain” of alerts. If a subsequent alert occurs within the coupling interval of any of the previous coupling intervals, that subsequent alert is also part of the chain.

Sequential coupling intervals in the chain are counted as a true positive 100 as long as one or more of the coupling intervals encompass a seizure. For example, in the illustrated example, the seizure occurs within the coupling window of the sixth alert of the second chain. Since each of the coupling intervals in the chain overlapped with a previous coupling window, and the overlapping coupling intervals extend back to the coupling window in the first alert in the chain, the entire chain of coupling intervals may be considered to be a single [symbol] (consolidated statistic). For some metrics, the chain of alerts and coupling intervals can be considered to be twelve separate single-sample TPs. Similar to the embodiments of FIG. 5A, pro-ictal intervals that are longer than an individual coupling window are able to be accommodated through the use of the chain of alerts.

As shown in FIG. 6, a false positive 110 occurs if a seizure does not occur within any of the coupling intervals in the chain of classifier alerts and coupling intervals. Similar to the TP metric, the entire chain of alerts and coupling intervals may be considered to be a single [symbol] (consolidated statistic), or seven individual FPs.

A TN occurs when no alert or coupling window is present during an interval that does not encompass a seizure. FIG. 6 illustrates two sets of TNs (5 TNs and 13 TNs). A single [symbol] (not shown) would occur if a seizure is not encompassed by a coupling interval.

Sensitivity (Sn), specificity (Sp), negative predictive value (NPV), and positive predictive value (PPV) using the methods of FIG. 6 are defined slightly differently from the methods of FIGS. 5A-5C. The methods of FIG. 6 use both the consolidated counts ( [symbol] [symbol] ) as well as the single sample counts (TN, TP, FN, FP):

$$\begin{matrix}{S}_{n} = \frac{\hat{T P}}{\hat{T P} + \hat{F N}} & ( 1 {a}^{′} ) \\ {S}_{p} = \frac{T N}{T N + F P} & ( 1 {b}^{′} ) \\ N P V = \frac{T N}{T N + F N} & ( 1 {c}^{′} ) \\ P P V = \frac{\hat{T P}}{\hat{T P} + \hat{F P}} & ( 1 {d}^{′} )\end{matrix}$$

The alert duration is characterized by the time between the first classifier alert generating a TP and seizure onset. A false alert duration is characterized by the time between the first false positive classifier alert and the end of the coupling interval of the last coupling interval in the chain, i.e., when the classifier output ceases showing a pro-ictal state before a seizure has occurred. Once again, a false alert indication may be useful in a seizure advisory algorithm. The false alert percentage and alert percentage may be calculated as:

$$\begin{matrix}False Alert Percentage ≅ \frac{F P}{T P + F P + T N + F N} = \frac{F P}{number of samples} & ( 2 ' ) \\ Alert Percentage ≅ \frac{T P + F P}{T P + F P + T N + F N} = \frac{T P + F P}{number of samples} & ( 2 '' )\end{matrix}$$

While the remaining discussion on statistical metrics is directed toward the methods embodied by the embodiments of FIGS. 5A-5C, such statistical methods may also be applicable to the methods of FIG. 6. Additionally, other combinations of point-wise and consolidated counts are possible. Furthermore, these methods can also be generalized to characterize the performance of an indicator that is used to indicate when the patient has a low susceptibility to having a seizure.

### Design for Patient Preference

The most commonly used metrics in the seizure prediction literature for quantification of algorithm performance are sensitivity and false positive rate (number of false positive warnings per unit time). Interestingly, these choices are not independent; sensitivity may be improved by allowing the false positive rate to degrade and vice versa. This complementary relationship between sensitivity and false positive rate may permit the algorithm to be adjusted to meet the needs of a particular patient, such as, for example, reducing the false positive rat) by reducing the sensitivity of the algorithm.

A common criterion is to fix the false positive rate (expressed in false positives per unit time), then measure and compare sensitivity. This is carried out by fixing a performance metric using in-sample data as part of training while actual performance is measured out-of-sample. While offering simplicity, this criterion may not be acceptable to all patients. For example, a subject experiencing one seizure per week may find that a false positive rate of one per week provides an attractive option: exchange one false alarm for one correctly anticipated seizure coupled with relative freedom from anxiety for the rest of the week. A subject experiencing one seizure every two months, however, will probably be far less enthusiastic about trading eight false alarms for each anticipated seizure.

An alternative approach used by one embodiment of the present invention is to match one performance metric to a patient-dependent characteristic, for example match the false positive rate to a multiple of the patient's seizure rate. By following this general rule, all subjects receive a similar benefit regardless of seizure frequency. This technique may also be used to match other performance metrics to a patient-dependent characteristic, e.g. percent time in warning or percent time in false warning.

### Statistical Validation

It is generally impossible to reproduce the results of real-world experiments precisely. This is particularly true of medical experiments, in which outcomes may be influenced by numerous factors beyond the control of the experimenter: demographic and physical differences between enrolled patients, confounding elements of daily life such as stress, comorbidity, environmental variation, etc. Accordingly, when two experimental alternatives are compared, it is desirable to ask whether observed performance differences are the result of real differences between the alternatives, or just the consequence of natural experimental variation. This question is answered by a statistical test that determines the probability of obtaining the observed performance difference (or greater) as a result of normal experimental variation under the assumption that no real difference exists between the two alternatives. This probability is referred to as the “p-value.”

Statistical tests may be divided into three categories: parametric, non-parametric, and numerical. Parametric tests, such as Student's t-test, are commonly used in the medical literature, and their popularity is due at least in part to their relative ease of computation and wide availability in statistical software. Unfortunately, parametric tests make assumptions about the nature of experimental variation that are sometimes violated in practice, creating the potential for incorrect conclusions. As a consequence, experiments and their metrics should be carefully designed and results examined to ensure that the assumptions are justified; a level of rigor that is rarely observed.

Non-parametric methods are enjoying increasingly popularity in the literature. They require more computational effort than parametric tests, but offer the advantage of removing a priori assumptions about experimental variation; rather, the statistical model is based on the data itself. These techniques are more robust than parametric tests, but typically have less power to detect small performance differences, making them a rigorous and conservative alternative.

The numerical approach to statistical testing utilizes computers to simulate thousands of experiment repetitions. These Monte Carlo simulations can handle very complex metrics and experimental designs, but often rely on custom written software specific to the experiment at hand. Thus the statistical test software itself should be validated for correctness.

The present invention has taken the approach of using metrics and experiments for which non-parametric statistical tests may be employed, but as can be appreciated, alternative embodiment could employ parametric or numerical statistical tests.

### Comparing a Seizure Prediction Algorithm to Chance for a Single Patient

In order to demonstrate that a seizure prediction algorithm is truly predictive, the seizure prediction algorithm is compared to a chance predictor. The block-wise model is utilized for this example. If the outputs of the algorithm were generated by chance, for any given dataset the probability of any particular scoring window being an alert window can be approximated by the proportion of alert windows to all scoring windows:

$$p = probability of positive = \frac{number of positives}{number of prediction windows} \qquad ( 3 )$$

A chance predictor according to the probability calculated in Equation 3 will generate the same proportion of alert time (true or false) as the seizure prediction algorithm.

For the chance predictor to score a true positive, the scoring window immediately preceding a seizure should have a positive output. The count of such windows is equivalent to a binomial counting process with probability p, with the number of trials equal to the number of seizures. The probability of k true positives is then given by:

$$\begin{matrix}P [ k true positives ] = {P}_{B} ( k , {n}_{s} , p ) = \frac{{n}_{s} !}{k ! ( {n}_{s} - k ) !} {{p}^{k} ( 1 - p )}^{{n}_{s} - k} & ( 4 ) \\ where & \\ {n}_{s} = number of seizures & ( 5 )\end{matrix}$$

The expectation value for TP is then

$$E [ T P ] = {\sum }_{k = 0}^{{n}_{s}} k \cdot {P}_{B} ( k , {n}_{s} , p ) = p \cdot {n}_{s} \qquad ( 6 )$$

yielding a sensitivity equal to

$${S}_{nc} ≡ E [ S n ( chance ) ] = \frac{E [ T P ]}{{n}_{s}} = p \qquad ( 7 )$$

The sensitivity difference between the candidate prediction algorithm and a corresponding chance predictor is obtained by combining equations (1a), (3), and (7):

$${S}_{n} - {S}_{nc} = \frac{T P}{T P + F N} - \frac{number of positives}{number of prediction windows} \qquad ( 8 )$$

which is seen to be just the difference between observed sensitivity and the percentage of time in warning. The derivation of p-value for an individual patient as shown below works for both the point-wise and block-wise methods. These equations are expressed in terms of the expected value of the chance predictor, Snc. The only difference between the point-wise and block-wise methods is in the calculation of Snc, which is covered elsewhere. In addition to population-based statistics, it is possible to test algorithm sensitivity versus chance for an individual patient. This approach yields an objective measure of the efficacy of an algorithm and can be used to quantify the affect on the ability of the algorithm to provide useful information of a change to algorithm to change the time spent in warning.

Consider an algorithm that identifies n of N seizures (i.e., sensitivity Sn=n/N). The two-sided p-value is the probability of observing a difference |n/N−Snc| or greater if the algorithm under evaluation is not different from chance, i.e.:

$$\begin{matrix}p = P [ ( {S}_{n} - {S}_{nc} ) \geq \vert \frac{n}{N} - {S}_{nc} \vert ] + P [ ( {S}_{n} - {S}_{nc} ) \leq - \vert \frac{n}{N} - {S}_{nc} \vert ] \\ = \{ \begin{matrix}P [ N \cdot {S}_{n} \geq n ] + P [ N \cdot {S}_{n} \leq ( 2 N \cdot {S}_{nc} - n ) ] , for \frac{n}{N} \geq {S}_{nc} \\ P [ N \cdot {S}_{n} \geq ( 2 N \cdot {S}_{nc} - n ) ] + P [ N \cdot {S}_{n} \leq n ] , for \frac{n}{N} < {S}_{nc}\end{matrix}\end{matrix} \qquad ( 9 )$$

Each of the N seizures can be considered a Bernoulli trial with the probability of prediction equal to the expected sensitivity of the chance predictor. Accordingly, Equation (9) can be rewritten in terms of the binomial cumulative distribution function

$$\begin{matrix}p = \{ \begin{matrix}[ 1 - {F}_{B} ( n - 1 ; N , {S}_{nc} ) ] + {F}_{B} ( {k}_{f} ; N , {S}_{nc} ) ] , for \frac{n}{N} \geq {S}_{nc} \\ [ 1 - {F}_{B} ( {k}_{c} ; N , {S}_{nc} ) ] + {F}_{B} ( n ; N , {S}_{nc} ) , for \frac{n}{N} \geq {S}_{nc}\end{matrix} & ( 10 ) \\ where & \\ {F}_{B} ( k ; n , p ) ≡ {\sum }_{j = 0}^{k} {f}_{B} ( j ; n , p ) & ( 11 ) \\ {f}_{B} ( k ; n , p ) ≡ ( \begin{matrix}n \\ k\end{matrix} ) {{p}^{k} ( 1 - p )}^{n - k} & ( 12 ) \\ and & \\ {k}_{f} = floor ( 2 N \cdot {S}_{nc} - n ) \\ {k}_{c} = ceiling ( 2 N \cdot {S}_{nc} - n ) & ( 13 )\end{matrix}$$

The first term of Equation 10 comprises the one-sided p-value for superiority of the algorithm compared to chance. Of particular note is the observation that the second term in (10) will contribute to the p-value only when expected sensitivity of the chance predictor is at least half that of the algorithm under test.

As an example, consider the following algorithm results for a patient with 5 seizures during a 138 hour observation period (92 scoring windows, 25 of which were positive)):

N=5 seizures

n=4 seizures predicted successfully

S_{nc}=25/92=27.2%

Hence the p-value is

p=1−F_{B}(3;5,0.272)=0.022

In the above result, the second term of (10) does not contribute, since S_{nc} is less than half of the observed sensitivity (k_{f} evaluates to a negative number). The algorithm has outperformed a chance predictor producing the same proportion of time in warning (27.2%) with sensitivity of 80% versus 27.2%, p=0.022. These results have been verified via Monte Carlo simulation.

It should be appreciated that equations 9-13 are applicable to any chance predictor for which the expected sensitivity can be calculated, given the characteristics of a corresponding algorithm under test.

### Comparing Two Seizure Prediction Algorithms Over A Population

The performance of any classifier and/or prediction algorithm can be modified by trading performance of one characteristic against a complementary one, i.e. determining the operating point. In FIG. 3 for example, sensitivity may be improved by using threshold (c) instead of threshold (a), but the percentage of time spent in alert will also increase. As was seen in the example of the predictor based on numbered slips of paper drawn from a hat, this trade-off is also available for chance predictors. As has been previously discussed, the “best” operating point depends upon patient preference. Thus to compare two different algorithms, it is necessary to first fix one performance characteristic according to patient preference, then compare another complementary characteristic. In the following example, the percentage of time in warning will be fixed, and the algorithm sensitivity compared.

This is carried out by determining an in-sample operating point so as to fix the percentage of time in warning at a desirable value. It must be recognized, however, that the actual percentage of time in warning (measured out-of-sample) will differ somewhat from the desired value (fixed in-sample) and from one algorithm to another, and is therefore only approximately “fixed.” Consequently, it is desirable to identify a test metric that corrects for the sensitivity advantage, owing entirely to chance, of an algorithm having a higher proportion of time in warning. This is done simply by calculating Sₙ−S_{nc} for each algorithm prior to comparison. For the block-wise model this is just equal to observed sensitivity minus the proportion of time in warning.

Sₙ−S_{nc} is calculated for each algorithm and for every patient in the population. Since both candidate algorithms are applied to every patient, a paired test should be used. The test metric is the difference in Sₙ−S_{nc} (the algorithm's advantage over chance) between the two algorithms computed on a patient-by-patient basis. The null and alternative hypotheses are:

H0:median(Sₙ−S_{nc} of algorithm #1−Sₙ−S_{nc} of algorithm #2)=0,

H1:median(sensitivity of algorithm #1−sensitivity of algorithm #2)≠0.

A 2-sided test is indicated by the alternative hypothesis, for which the non-parametric Wilcoxon signed-rank test is appropriate.

### Out-of-Sample Testing

When evaluating a classification algorithm, independence of testing and training data is often ensured by separating the data into independent training and testing partitions, the latter referred to as a “hold-out.” The algorithm is trained using only the data in the training partition, and characterized by applying it without modification to the testing partition.

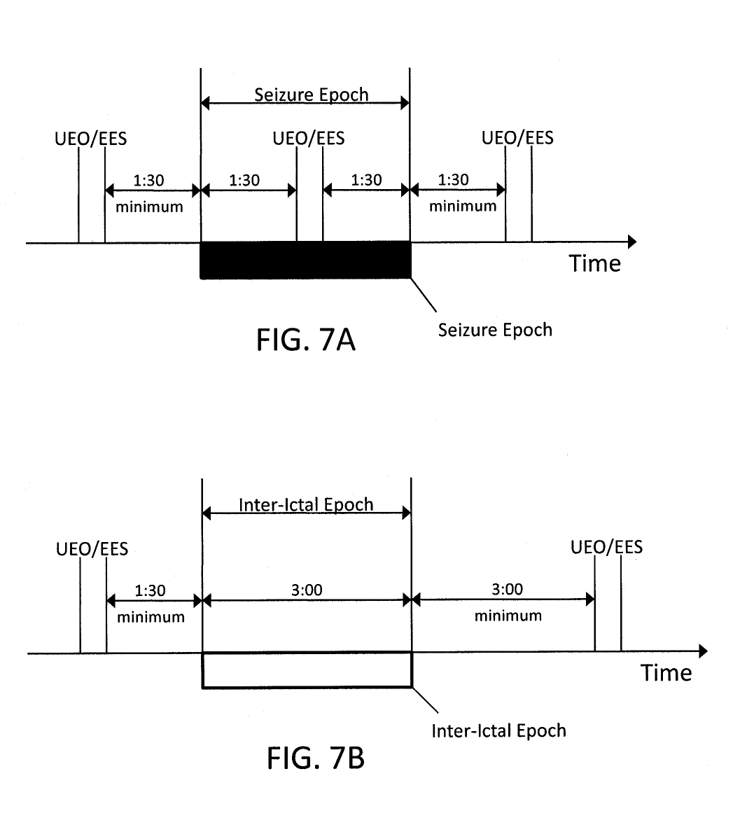

Care should be exercised in partitioning the data for evaluation of a seizure advisory algorithm, since the input data is not a set of statistically independent events, but rather a time series of measurements with short-term correlations. Consequently, the hold-out cannot be partitioned on a sample-by-sample basis (e.g. every other sample is used for training and the alternates for testing), but should be done over longer time epochs. Some desirable attributes of the data epochs that may be used by the present invention are shown in FIGS. 7A and 7B and described as follows:
Seizure epochs are shown in FIG. 7A and typically contains a single seizure that is well separated from other seizures, and are assumed to be statistically independent events. Seizure epochs are at least 3 hours in duration (plus the duration of the seizure)—which includes at least one hour and thirty minutes prior to the unequivocal electrographic onset (UEO) and one hour thirty minutes after the electrographic end of the seizure (EES). Such seizure epochs are typically separated in time by at least about one hour and thirty minutes from a prior seizure. The time separation can be longer (or shorter) as desired, and the time separation from prior seizures and subsequent seizures may be the same (e.g., three hours) or different.
Interictal epochs are illustrated in FIG. 7B and are typically 3 hours in duration, and are well separated from seizure activity. In one preferred embodiment, the interictal epochs are separated by at least one hour and thirty minutes after a prior seizure and separated at least three hours from a subsequent seizure. If desired, the time separation can be longer (or shorter) as desired, and the time separation from prior seizures and subsequent seizures may be the same (e.g., three hours) or different.
Data that is not assigned to either a seizure or interictal epoch is instead assigned to an epoch labeled “other”.

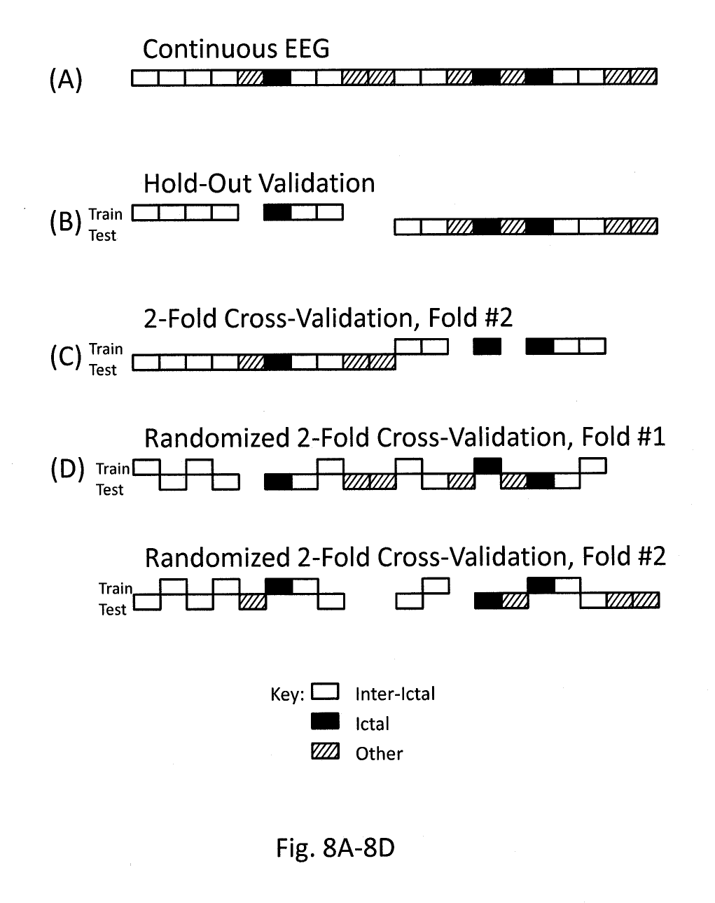

FIGS. 8A-8D illustrate the interictal epochs (white box), seizure epochs (dark box), and “other” epochs used for testing (diagonal hatched box). The seizure and interictal epochs defined in this manner can be used for either training or testing. Since the “other” epochs may contain statistically dependent events, e.g. multiple closely spaced seizures, they are used for testing only. A schematic representation of a hold-out validation using epochs is illustrated in FIGS. 8A and 8B.

One disadvantage of hold-out validation is that only half of the available data is utilized in calculating algorithm performance. This is exacerbated by the fact that many EMU patients experience only a small number of seizures during their EMU visit. For example, if a patient experiences only three seizures and two are retained for the hold-out, then only three sensitivity results are possible: 0%, 50%, or 100%. Such coarse granularity adds variance to the sampling distribution of the experiment, making it much more difficult to recognize significant results.

The available data may be better utilized if the roles of the training and hold-out partitions are reversed in order to test the other half of the data (FIG. 8C). By combining results of the reversed hold-out with the original (FIGS. 8B and 8C), a continuous record of test results is obtained, allowing metrics to be calculated over the entire data record, rather than just half. This technique is referred to as a 2-fold cross-validation.

Cross-validation may be further improved by randomizing the assignment of epochs to the training and test partitions in order to distribute possibly confounding events, e.g. cyclical neurological states, circadian cycles, states of vigilance, across the training and holdout partitions (FIG. 8D).

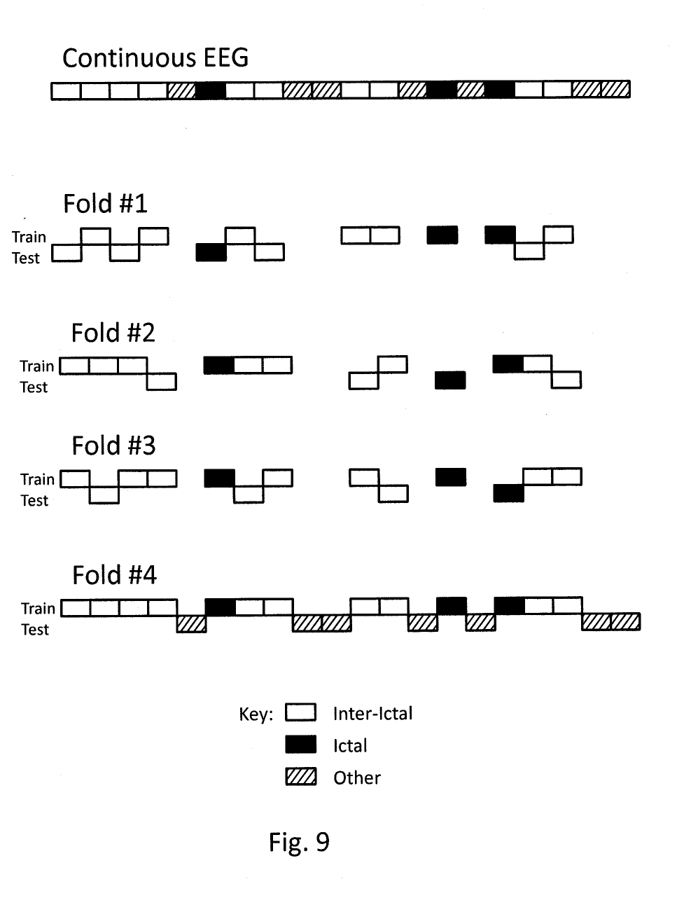

While two-fold cross-validation allows algorithm metrics to be calculated over the entire data record, only 50% of the data can be used for training within a single fold. As shown in FIG. 9, this percentage may be increased by utilizing k-fold cross-validation (where “k” represents the number of seizures), in which the hold-out for each fold contains only one seizure epoch and a corresponding proportion of interictal epochs. In this manner, the majority of available data can be used for training within each fold, while testing is still performed on an independent out-of-sample set of epochs.

It should be noted that the “other” epochs are generally not used for training purposes. In order to evaluate the “other” epochs, an extra fold (Fold #4 in FIG. 9) is added to the usual leave-one-out paradigm. The training partition of the extra fold is comprised of all seizure and interictal epochs, while the testing hold-out contains all “other” epochs.

The methods and systems of one embodiment of the present invention use leave-one-out cross-validation, with randomized assignment of data epochs to the test and training partitions of each fold. While FIG. 9 shows a k-fold cross validation, it should be appreciated that any number of folds may be used with the methods and systems of the present invention.

To place bounds on estimation error, multiple cross-validations may be performed. Each cross-validation uses a different randomization of epoch assignments for the various test and training partitions across the cross-validation folds. Finally, the results of the leave-one-out cross-validations may be compared to the results of a simple 2-fold cross-validation. Furthermore, other techniques may be used, such as a progressive hold-out.

A number of common methodological and statistical weaknesses that have been identified in the seizure prediction literature, and partially catalogued by Mormann and colleagues [Mormann et al. 2006a] have been alleviated by the methods and systems of the present invention.

A Seizure Advisory System (SAS) is used to carry out the methods described above. One embodiment of the SAS utilizes two feature calculations applied uniformly to all electrode contacts of all patients in the database described above. Classifiers are patient specific, primarily serving to identify the electrodes that contain information relevant to seizure prediction. As described above, classifier design is performed using data belonging to time epochs that are independent of the epochs used for evaluation of algorithm performance. Classification results are produced at intervals of 1 second, corresponding to more than 600,000 classifications during a 7-day visit to the EMU.

### Data Analysis

The results described below were achieved using the block-wise method shown in FIGS. 5A-5C. The block size adopted for the results described in these examples is 90 minutes (the scoring window), with a one minute detection window to ensure that seizure detection is not confused with seizure prediction. Of course, any length block-size could be used. Each scoring window is labeled as an “alert window” if it contains one or more classifier alerts and a “non-alert window” if there are no classifier alerts whatsoever. Metrics are reported as median [lower quartile−upper quartile] unless otherwise specified.

The accuracy of SAS for predicting impending seizure (sensitivity) is the primary observation, but other observations regarding the performance may be measured. In addition to sensitivity, the distribution of alert durations is reported. Predictive ability is tested under the null and alternative hypotheses

H₀:median(sensitivity of SAS−sensitivity of chance predictor)=0,

H₁:median(sensitivity of SAS−sensitivity of chance predictor)≠0.

The chance predictor is computed so as to produce the same proportion of alert windows as the SAS.

A 2-sided test is indicated by the alternative hypothesis, for which the non-parametric Wilcoxon signed-rank test is employed.

Sensitivity is measured and reported under two different conditions:
1. With up to one false alert window allowed per seizure,
2. With up to two false alert windows allowed per seizure.

These conditions predetermine the specificity, false positive rate, and percentage of time spent in false alert as a function of each patient's seizure rate in the EMU. As a consequence, these metrics must be interpreted as conditions of the protocol rather than results of the experiment. Percentage time spent in false alert is reported as the primary measure of interest.

Algorithm testing is performed on data that is independent of the data used for algorithm training. This is accomplished by dividing the EEG record into epochs of approximately 3 hours duration (FIGS. 7A and 7B). A k-fold cross validation is performed in which each fold contains one seizure epoch, and a proportional share of randomized interictal epochs. The randomization serves to distribute confounding cyclical events (circadian cycles, sleep state, etc.) across all folds of the cross-validation. A simple 2-fold cross-validation is also performed to demonstrate consistency with a conventional holdout strategy. (FIGS. 6A-6D). Epoch definitions are independent of the block-wise statistical approach—they are used to identify training and test data.

Cluster seizures present a special scoring challenge since they are not statistically independent events. Within a cluster of seizures, a relatively simple ictal or postictal detector will appear to perform well as a “predictor” for the subsequent seizure. To avoid this ambiguity, only the initial seizure of a cluster is used for calculating algorithm sensitivity.

No distinction is made between clinical or sub-clinical seizures. Nor are seizures differentiated by seizure duration or intensity—all annotated seizures are treated equally.

### Feature Outputs

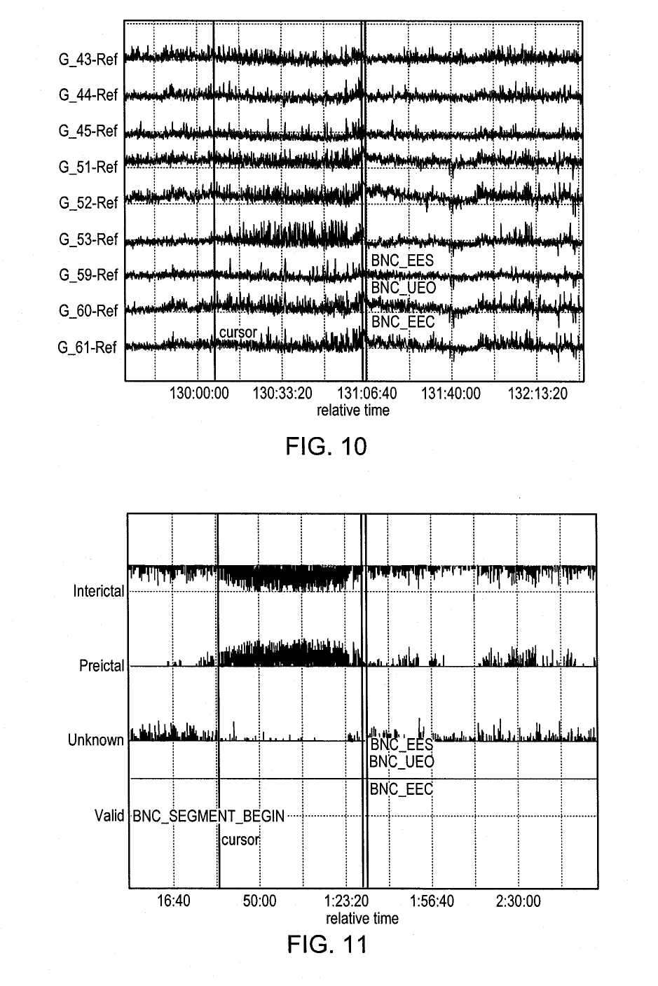

FIG. 10 illustrates nine traces that represent feature values calculated from a three-by-three section of a subdural electrode grid located over the origin of seizure activity. Large changes in feature behavior are clearly noticeable 60 minutes prior to the seizure.

### Classifier Outputs

Once features have been calculated, they are analyzed by a mathematical classifier in order to determine whether the data is representative of interictal or pro-ictal behavior. FIG. 11 shows the classification results from the data of FIG. 10. Each trace represents a particular class probability ranging from zero to one, with the classifier distinguishing between interictal, pro-ictal and “unknown” classes. Incorporation of an “unknown” class captures observations that are dissimilar from the information used to train the classifier, and serves to reduce the error rates for the known classes. It is clear from FIG. 11 that the probability of belonging to the pro-ictal class can be used as an excellent seizure predictor, with outputs prior to 90 minutes before the seizure essentially equal to zero, and intermittently increasing to unity as the seizure is approached. This intermittent behavior is observable in both the feature and classifier outputs, and is typical of the evolution toward seizure.

### Alert Signals

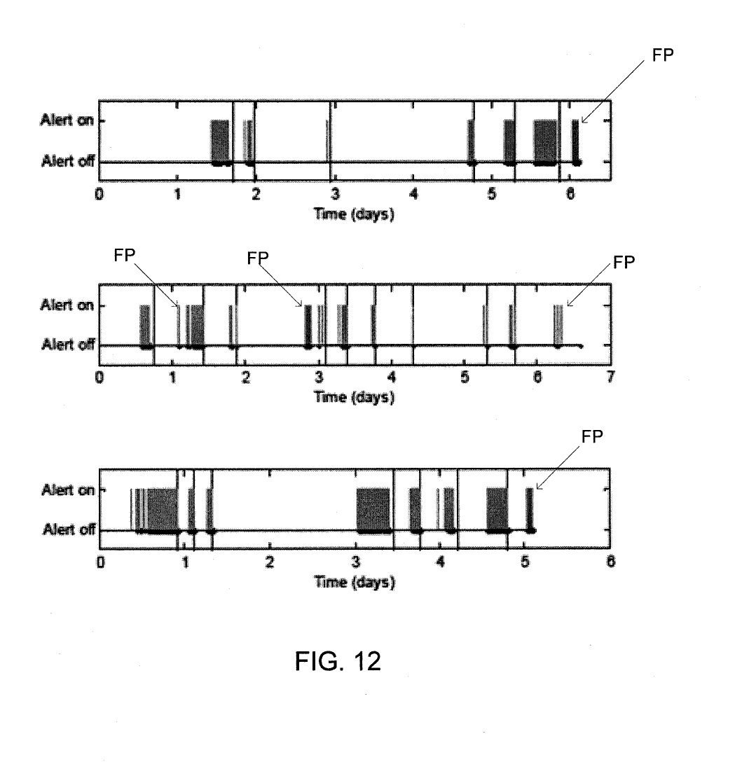

Alert signals result from applying a threshold to the output of a classifier, e.g., by issuing an alert of increased susceptibility to seizure whenever the probability of belonging to the pro-ictal class exceeds a fixed percentage. FIG. 12 illustrates alert signals for three different patients over the course of their EMU stay. Of particular note are the long intervals (days) without false positives, the variability of alert duration, and the non-random temporal distribution of false positives. These effects are considered in more detail below.

### Sensitivity and Alert Duration

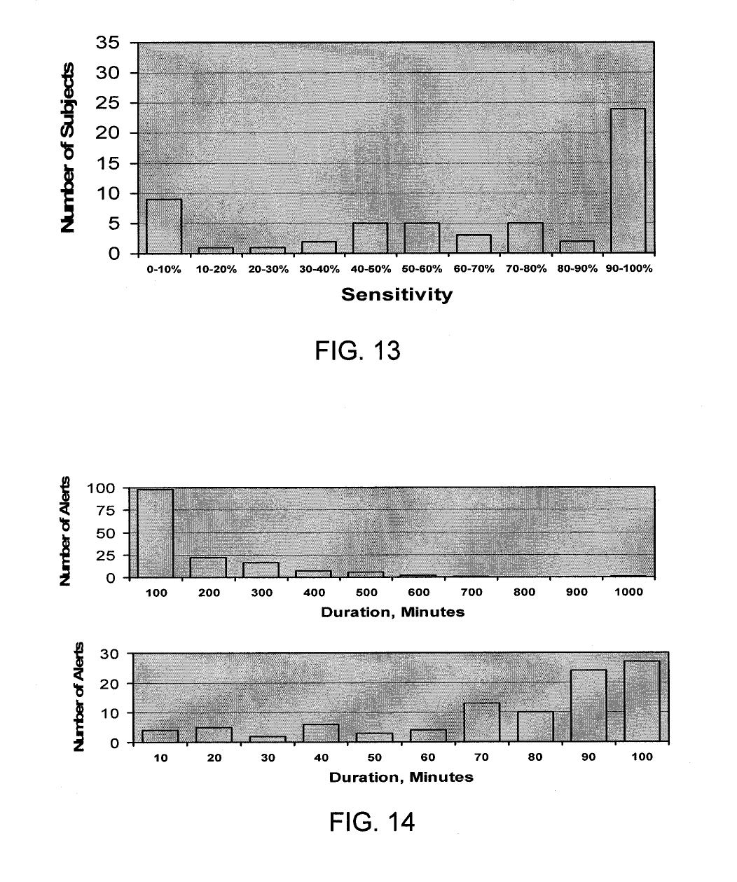

With a maximum of one false positive allowed per seizure, the distribution of sensitivities across the population is illustrated in FIG. 13 (median 75% [50%-100%], p<0.0001 vs. chance predictor). It is of particular note that 42% of patients have sensitivity of 100%. Of the 9 patients with zero percent sensitivity, 5 patients did not have any surface recording electrodes in the EMU, i.e., they had depth electrodes only—a disproportionate share, suggesting that surface recording electrodes may provide optimal performance.

Alert duration is 91 [71-181] minutes, with a minimum of 3.7 minutes. The distribution is approximately log-normal, as can be appreciated from FIG. 14.

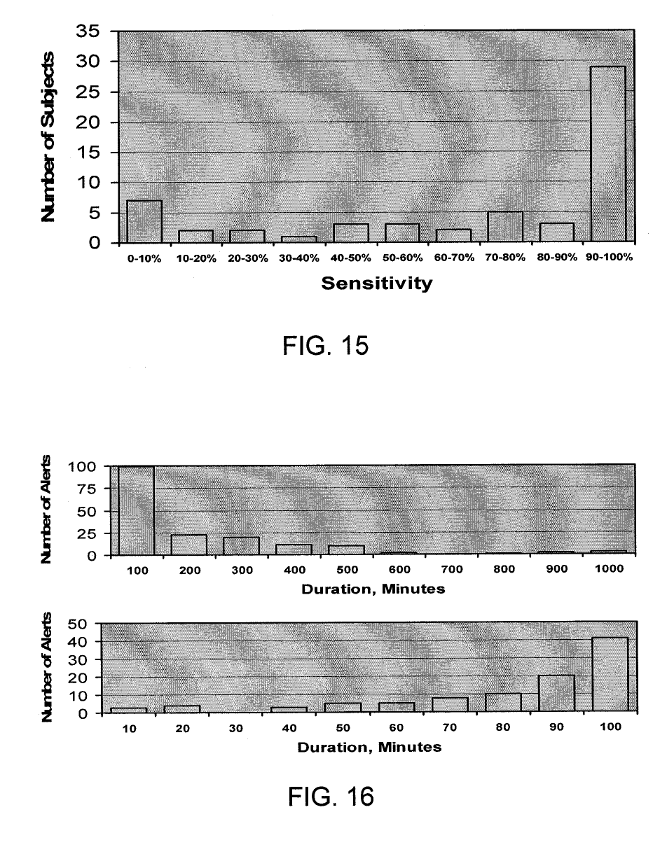

With a maximum of two false positives allowed per seizure, the distribution of sensitivities across the population is illustrated in FIG. 15 (median 100% [50%-100%], p<0.0001 vs. chance predictor). In this case, 51% of patients have sensitivity of 100%, and 3 of 7 patients with zero percent sensitivity have depth electrodes only.

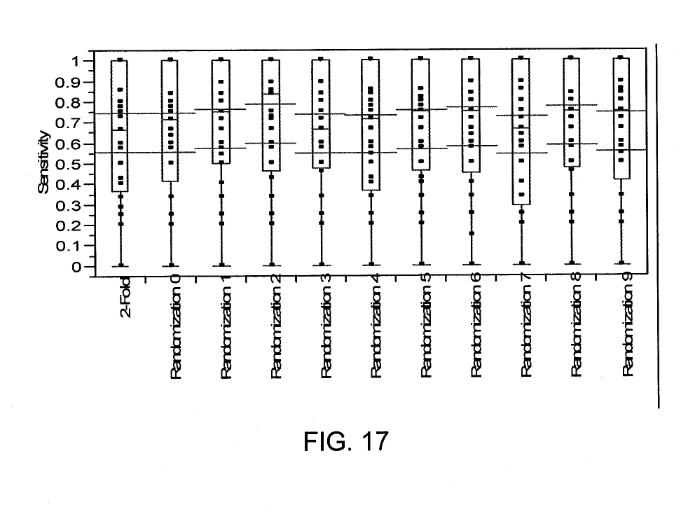

The distribution of alert durations remains essentially unchanged at 91[84-256] minutes (minimum 3.7 minutes) in spite of the number of anticipated seizures increasing from 153 to 171 (FIG. 16).

### Percentage of Time in False Alert

The percentage of time spent in false alert is essentially determined by a patient's seizure rate in the EMU, resulting from the protocol requirement of matching the number of false positives to the number of seizures. It must consequently be viewed as a condition of the experiment rather than a result. It is important to note that this metric is calculated based on classifier outputs rather than a specific user interface, and is therefore an approximation of achievable performance.

With these caveats in mind, the median percentage of time spent in false alert is 15% [8%-22%] with one false positive allowed per seizure, and 18% [12%-34%] with two false positives allowed per seizure. The small difference between the two allowable false positive rates is attributable to the 100% sensitivity achieved by many patients with fewer false positives than seizures. The translation of these results to the outpatient environment is considered below.

### Cross-Validation Results

Multiple cross-validation epoch randomizations were performed to test for confounding of cyclical neurological events (e.g. sleep state, circadian cycles, etc.). The results are shown in FIG. 17. Conventional 2-fold cross-validation utilizing 50% data holdouts is also shown for comparison. All cross-validations produced equivalent results (p=0.998, Kruskal-Wallis test).

### Sensitivity

The sensitivity data presented here is based on very broad inclusion criteria: patients must have at least 2 well-isolated electrographic seizures and 6 hours of interictal data collected in the EMU. There are no exclusion criteria that have been applied. As a consequence, the dataset includes patients with generalized seizure onset, multi-focal epilepsy, and patients whose epileptogenic focus is not covered by a surface recording electrode. While the dataset is dominated by patients with temporal lobe epilepsy, it also includes parietal and frontal lobe patients. Patients may have cortical surface electrodes only, depth electrodes only, or both. Electrode contact counts range from 4 to 36 for depth and 20 to 144 for cortical. Finally, sensitivity is scored against all seizures, clinical and sub-clinical, regardless of intensity or brevity.

Two types of electrodes are typically used in EMU studies: cortical electrodes and depth electrodes. In the dataset used herein, 13 of 57 patients have depth electrodes only. These patients exhibit much lower sensitivity than those having cortical surface electrodes only (median 40% vs. 100%, p=0.003). If these patients are excluded from the dataset, overall prediction sensitivity (one false positive allowed per seizure) increases from 75% to 94% median (n=44). Thus, in this dataset we found that the presence of depth electrodes did not improve results. This finding may be attributable to the more localized nature of signals collected by depth electrodes, or to selection bias inherent in the decision to place only depth electrodes in a particular patient.

With regards to the number of cortical surface electrodes needed for seizure prediction, regression analysis indicates that sensitivity is independent of the number of surface electrodes (p=0.999). Thus 20 electrodes (the minimum count in the dataset) appears to be more than adequate for prediction purposes. Further subgroup analysis is expected to reveal additional insights, and may also provide opportunities for identification of non-predictive patients for screening purposes.

### False Positives and Percentage of Time in False Alert

The reported false alert times reported herein correspond to false positive rates of 0.10 to 0.12 per hour, comparing favorably with the best results reported in the seizure prediction literature. {Mormann et al. 2006} Furthermore, the total time spent in false alert is on par with the combined time spent in seizure and consequent postictal neural-suppression.

An open and compelling question is how the rate of false positive alerts will translate to the outpatient environment. If false positives are comprised of random classification errors, e.g., caused by noise, then the outpatient false positive rate may not be significantly different from that observed in the EMU.

An alternative possibility has been raised, however, by a recent study that described the preictal state as “a stochastic, probabilistic state out of which seizures might arise,” but from which a seizure is not inevitable[Wong et al. 2006] Under this interpretation, the “false positives” may actually be true detections of a pro-ictal state that for one reason or another (e.g. lack of an external precipitating event) returned to the interictal state rather than terminating in seizure. Under this scenario, the frequency of “false positives” is expected to decrease along with seizure frequency when the provocations of the EMU are removed. Evidence for this interpretation is provided by the temporal pattern of alert signals observed in this study (FIG. 12) in which the false positives are clustered tightly together, as would be the case for detection of a pro-ictal state, rather than distributed randomly over the duration of the EMU visit as would be the case for random classification errors.

### Ambulatory Seizure Advisory System

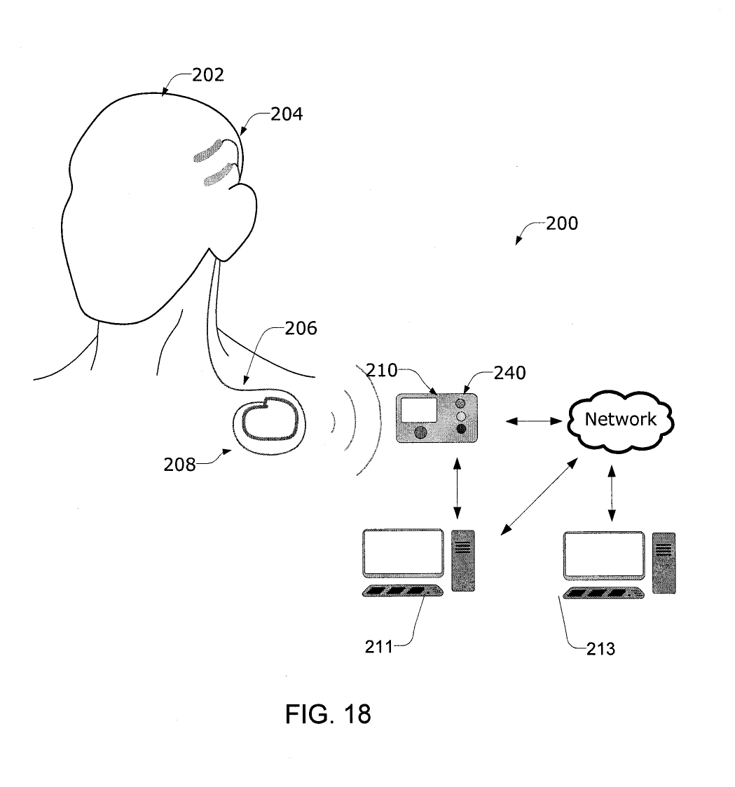

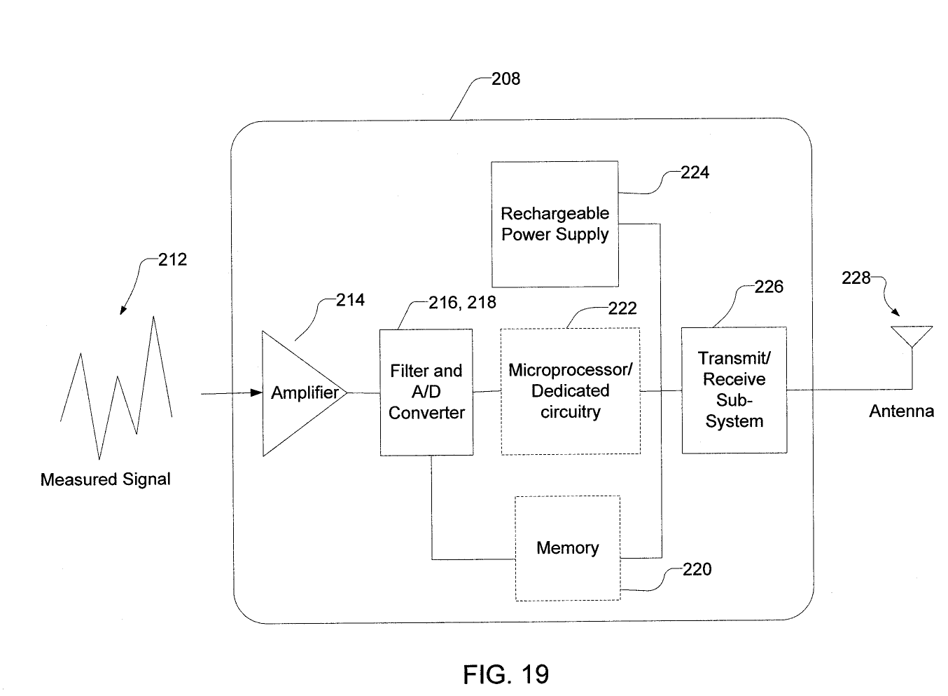

One or more of the methods described above may be used to develop a patient-specific seizure prediction algorithm. Once the algorithm has been trained on the patient training data and tailored to the particular patient, the seizure prediction algorithm may be embodied as one or more modules (e.g., stored in memory) in a seizure advisory system. FIG. 18 illustrates one embodiment of a system in which the aforementioned seizure advisory algorithms of the present invention may be embodied. The system 200 is used to monitor a patient 202 for purposes of measuring physiological signals and predicting neurological events. The system 200 of the embodiment provides for substantially continuous sampling of brain wave electrical signals such as in electroencephalograms or electrocorticograms, (referred to collectively as EEGs).

The system 200 comprises one or more sensors 204 configured to measure signals from the patient 202. The sensors 204 may be located anywhere on the patient 202. In the exemplary embodiment, the sensors 204 are configured to sample electrical activity from the patient's brain, such as EEG signals. The sensors 204 may be attached to the surface of the patient's body (e.g., scalp electrodes), attached to the head (e.g., subcutaneous electrodes, bone screw electrodes, etc.), or, preferably, may be implanted intracranially in the patient 202. In one embodiment, one or more of the sensors 204 will be implanted adjacent a previously identified epileptic focus, a portion of the brain where such a focus is believed to be located, or adjacent a portion of a seizure network.

Any number of sensors 204 may be employed, but the sensors 204 will typically include between 1 sensor and 20 sensors, and preferably between about 8 and 16 sensors. The sensors may take a variety of forms. In one embodiment, the sensors comprise grid electrodes, strip electrodes and/or depth electrodes which may be permanently implanted through burr holes in the head. Exact positioning of the sensors will usually depend on the desired type of measurement. In addition to measuring brain activity, other sensors (not shown) may be employed to measure other physiological signals from the patient 202.

In an embodiment, the sensors 204 will be configured to substantially continuously sample the brain activity of the groups of neurons in the immediate vicinity of the sensors 204. The sensors 204 are electrically joined via cables 206 to an implanted communication unit 208, but sensors 204 may also be leadless (not shown). In one embodiment, the cables 206 and communication unit 208 will be implanted in the patient 202. For example, the transponder unit 208 may be implanted in a subclavicular cavity of the patient 202. In alternative embodiments, the cables 206 and transponder unit 208 may be attached to the patient 202 externally.

In one embodiment, the communication unit 208 is configured to facilitate the sampling of signals from the sensors 204. Sampling of brain activity is typically carried out at a rate above about 200 Hz, and preferably between about 200 Hz and about 1000 Hz, and most preferably at about 400 Hz. The sampling rates could be higher or lower, depending on the specific conditions being monitored, the patient 202, and other factors. Each sample of the patient's brain activity is typically encoded using between about 8 bits per sample and about 32 bits per sample, and preferably about 16 bits per sample.

In alternative embodiments, the communication unit 208 may be configured to measure the signals on a non-continuous basis. In such embodiments, signals may be measured periodically or aperiodically.

An external data device 210 is preferably carried external to the body of the patient 202. The external data device 210 receives and stores signals, including measured signals and possibly other physiological signals, from the communication unit 208. External data device 210 could also receive and store extracted features, classifier outputs, patient inputs, etc. Communication between the external data device 210 and the communication unit 208 may be carried out through wireless communication. The wireless communication link between the external data device 210 and the communication unit 208 may provide a one-way or two-way communication link for transmitting data. In alternative embodiments, it may be desirable to have a direct communications link from the external data device 210 to the communication unit 208, such as, for example, via an interface device positioned below the patient's skin. The interface (not shown) may take the form of a magnetically attached transducer that would enable power to be continuously delivered to the communication unit 208 and would provide for relatively higher rates of data transmission. Error detection and correction methods may be used to help insure the integrity of transmitted data. If desired, the wireless data signals can be encrypted prior to transmission to the external data device 210.

FIG. 19 depicts a block diagram of one embodiment of a communication unit 208 that may be used with the systems and methods described herein. Energy for the system is supplied by a rechargeable power supply 224. The rechargeable power supply may be a battery, or the like. The rechargeable power supply 224 may also be in communication with a transmit/receive subsystem 226 so as to receive power from outside the body by inductive coupling, radiofrequency (RF) coupling, etc. Power supply 224 will generally be used to provide power to the other components of the implantable device. Signals 212 from the sensors 204 are received by the communication unit 208. The signals may be initially conditioned by an amplifier 214, a filter 216, and an analog-to-digital converter 218. A memory module 220 may be provided for storage of some of the sampled signals prior to transmission via a transmit/receive subsystem 226 and antenna 228 to the external data device 210. For example, the memory module 220 may be used as a buffer to temporarily store the conditioned signals from the sensors 204 if there are problems with transmitting data to the external data device 210, such as may occur if the external data device 210 experiences power problems or is out of range of the communications system. The external data device 210 can be configured to communicate a warning signal to the patient in the case of data transmission problems to inform the patient and allow him or her to correct the problem.

The communication unit 208 may optionally comprise circuitry of a digital or analog or combined digital/analog nature and/or a microprocessor, referred to herein collectively as “microprocessor” 222, for processing the signals prior to transmission to the external data device 210. The microprocessor 222 may execute at least portions of the analysis as described herein. For example, in some configurations, the microprocessor 222 may run the one or more feature extractors from the seizure prediction algorithm that extract characteristics of the measured signal that are relevant to the purpose of monitoring. Thus, if the system is being used for diagnosing or monitoring epileptic patients, the extracted characteristics (either alone or in combination with other characteristics) may be indicative or predictive of a neurological event. Once the characteristic(s) are extracted, the microprocessor 222 may transmit the extracted characteristic(s) to the external data device 210 and/or store the extracted characteristic(s) in memory 220. Because the transmission of the extracted characteristics is likely to include less data than the measured signal itself, such a configuration will likely reduce the bandwidth requirements for the communication link between the communication unit 208 and the external data device 210.

In some configurations, the microprocessor 222 in the communication unit 208 may run one or more classifiers (not shown) of the seizure prediction algorithm. The result of the classification may be communicated to the external data device 210.

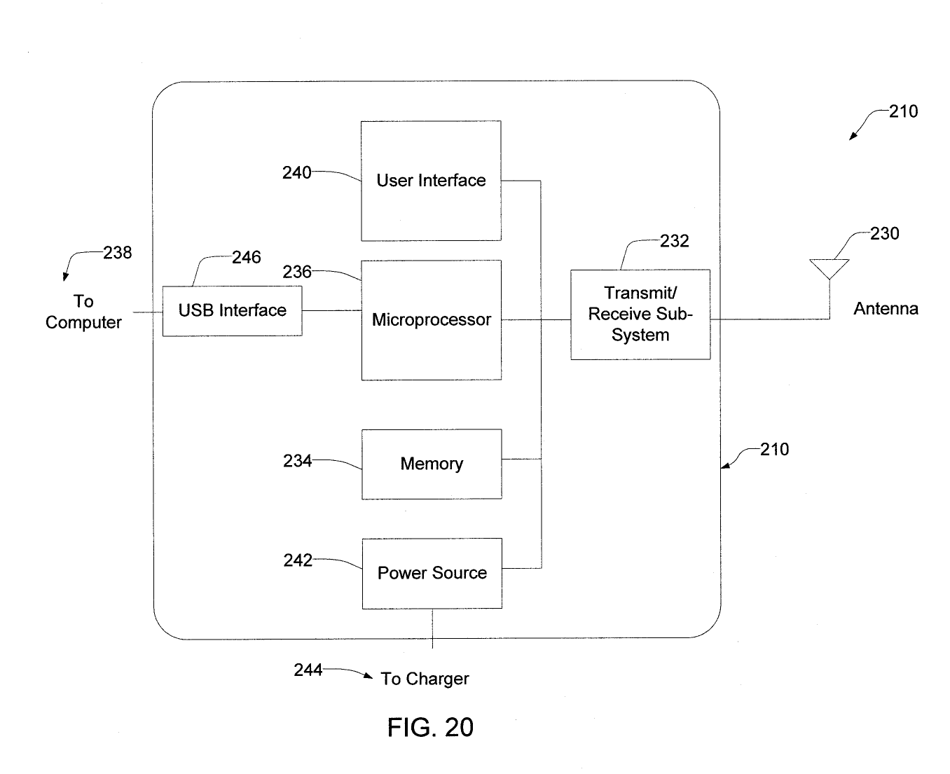

While the external data device 210 may include any combination of conventional components, FIG. 20 provides a schematic diagram of some of the components that may be included. Signals from the communication unit 208 are received at an antenna 230 and conveyed to a transmit/receive subsystem 232. The signals received may include, for example, a raw measured signal, a processed measured signal, extracted characteristics from the measured signal, a result from analysis software that ran on the implanted microprocessor 222, or any combination thereof.

The received data may thereafter be stored in memory 234, such as a hard drive, RAM, EEPROM, removable flash memory, or the like and/or processed by a microprocessor, application specific integrated circuit (ASIC) or other dedicated circuitry of a digital or analog or combined digital/analog nature, referred to herein collectively as a “microprocessor” 236. Microprocessor 236 may be configured to request that the communication unit 208 perform various checks (e.g., sensor impedance checks) or calibrations prior to signal recording and/or at specified times to ensure the proper functioning of the system.

Data may be transmitted from memory 234 to microprocessor 236 where the data may optionally undergo additional processing. For example, if the transmitted data is encrypted, it may be decrypted. The microprocessor 236 may also comprise one or more filters that filter out low frequency or high-frequency artifacts (e.g., muscle movement artifacts, eye-blink artifacts, chewing, etc.) so as to prevent contamination of the measured signals.

External data device 210 will typically include a user interface 240 for displaying outputs to the patient and for receiving inputs from the patient. The user interface will typically comprise outputs such as auditory devices (e.g., speakers) visual devices (e.g., LCD display, LEDs, etc.), tactile devices (e.g., vibratory mechanisms), or the like, and inputs, such as a plurality of buttons, a touch screen, and/or a scroll wheel. User interface 240 may include any number of types of outputs to indicate to the patient their brain state (sometimes referred to herein as a “brain state indicator”). In one preferred embodiment, the user interface 240 may indicate to the patient if they are in a contra-ictal state, a pro-ictal state, or an “other” brain state (e.g., not in a contra-ictal state or a pro-ictal state). An example of a useful brain state indicator are a green light when the patient is in contra-ictal state, a red light when the patient is in a pro-ictal state, and a yellow light when the patient is in the “other” brain state.

The user interface may be adapted to allow the patient to indicate and record certain events. For example, the patient may indicate that medication has been taken, the dosage, the type of medication, meal intake, sleep, drowsiness, occurrence of an aura, occurrence of a neurological event, or the like. Such inputs may be used in conjunction with the measured data to improve the analysis.

The LCD display may be used to output a variety of different communications to the patient including, status of the device (e.g., memory capacity remaining), battery state of one or more components of system, whether or not the external data device 210 is within communication range of the communication unit 208, a warning (e.g., a neurological event warning), a prediction (e.g., a neurological event prediction), a recommendation (e.g., “take medicine”), or the like. It may be desirable to provide an audio output or vibratory output to the patient in addition to or as an alternative to the visual display on the LCD.

External data device 210 may also include a power source 242 or other conventional power supply that is in communication with at least one other component of external data device 210. The power source 242 may be rechargeable. If the power source 242 is rechargeable, the power source may optionally have an interface for communication with a charger 244. While not shown in FIG. 20, external data device 210 will typically comprise a clock circuit (e.g., oscillator and frequency synthesizer) to provide the time base for synchronizing the external data device 210 and the communication unit 208.

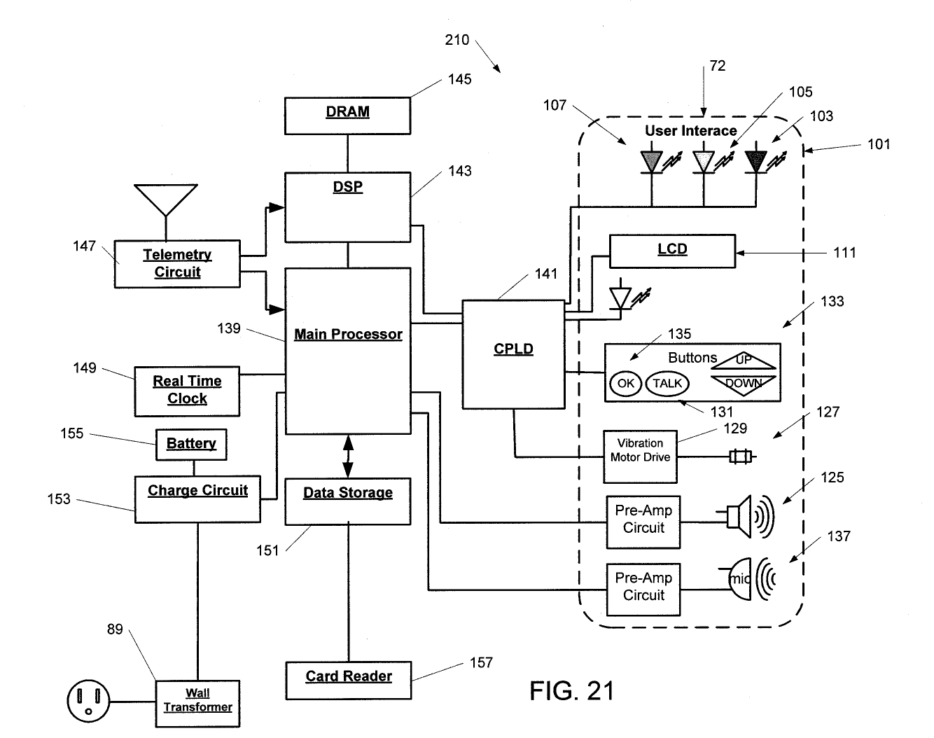

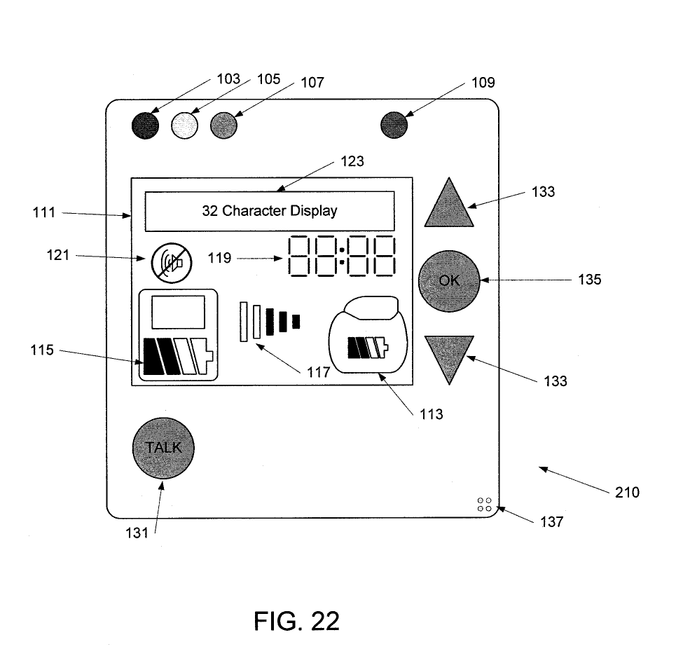

FIGS. 21 and 22 illustrate embodiments of an external assembly 210 for a seizure advisory system. External assembly 210 shows a user interface 72 that includes a variety of indicators for providing system status and alerts to the subject. User interface 72 may include one or more indicators 101 that indicate the subject's brain state. In the illustrated embodiment, the output includes light indicators 101 (for example, LEDs) that comprise one or more (e.g., preferably two or more) discrete outputs that differentiate between a variety of different brain states. In the illustrated embodiment, the brain state indicators 101 include a red light 103, yellow/blue light 105, and a green light 107 for indicating the subject's different brain states (described more fully below). In some configurations the lights may be solid, blink or provide different sequences of flashing to indicate different brain states. If desired, the light indicators may also include an “alert” or “information” light 109 that is separate from the brain state indicators so as to minimize the potential confusion by the subject.

External assembly 210 may also include a liquid crystal display (“LCD”) 111 or other display for providing system status outputs to the subject. The LCD 111 generally displays the system components' status and prompts for the subject. For example, as shown in FIG. 22, LCD 111 can display indicators, in the form of text or icons, such as, for example, implantable device battery strength 113, external assembly battery strength 115, and signal strength 117 between the implantable device and the external assembly 20. If desired, the LCD may also display the algorithm output (e.g., brain state indication) and the user interface 72 may not require the separate brain state indicator(s) 101. The output on the LCD is preferably continuous, but in some embodiments may appear only upon the occurrence of an event or change of the system status and/or the LCD may enter a sleep mode until the subject activates a user input. LCD 111 is also shown including a clock 119, audio status 121 (icon shows PAD is muted), and character display 123 for visual text alerts to the subject—such as an estimated time to seizure or an estimated “contra-ictal” time. While not shown in FIG. 21 or FIG. 22, the LCD 111 may also indicate the amount of free memory remaining on the memory card.

External assembly 210 may also include a speaker 125 and a pre-amp circuit to provide audio outputs to the subject (e.g., beeps, tones, music, recorded voice alerts, etc.) that may indicate brain state or system status to the subject. User interface 72 may also include a vibratory output device 127 and a vibration motor drive 129 to provide a tactile alert to the subject, which may be used separately from or in conjunction with the visual and audio outputs provided to the subject. The vibratory output device 127 is generally disposed within external assembly 20, and is described in more detail below. Depending on the desired configuration any of the aforementioned outputs may be combined to provide information to the subject.

The external assembly 210 preferably comprises one or more patient inputs that allow the patient to provide inputs to the external assembly. In the illustrated embodiment, the inputs comprise one or more physical inputs (e.g., buttons 131, 133, 135) and an audio input (in the form of a microphone 137 and a pre-amp circuit).

Similar to conventional cellular phones, the inputs 131, 133, 135 may be used to toggle between the different types of outputs provided by the external assembly. For example, the patient can use buttons 133 to choose to be notified by tactile alerts such as vibration rather than audio alerts (if, for example, a patient is in a movie theater). Or the patient may wish to turn the alerts off altogether (if, for example, the subject is going to sleep). In addition to choosing the type of alert, the patient can choose the characteristics of the type of alert. For example, the patient can set the audio tone alerts to a low volume, medium volume, or to a high volume.

Some embodiments of the external assembly 210 will allow for recording audio, such as voice data. A dedicated voice recording user input 131 may be activated to allow for voice recording. In preferred embodiments, the voice recording may be used as an audio subject seizure diary. Such a diary may be used by the subject to record when a seizure has occurred, when an aura or prodrome has occurred, when a medication has been taken, to record patient's sleep state, stress level, etc. Such voice recordings may be time stamped and stored in data storage of the external assembly and may be transferred along with recorded EEG signals to the physician's computer. Such voice recordings may thereafter be overlaid over the EEG signals and used to interpret the subject's EEG signals and improve the training of the subject's customized algorithm, if desired.

The one or more inputs may also be used to acknowledge system status alerts and/or brain state alerts. For example, if the external assembly provides an output that indicates a change in brain state, one or more of the LEDs 101 may blink, the vibratory output may be produced, and/or an audio alert may be generated. In order to turn off the audio alert, turn off the vibratory alert and/or to stop the LEDs from blinking, the patient may be required to acknowledge receiving the alert by actuating one of the user inputs (e.g., button 135).

External assembly 210 may comprise a main processor 139 and a complex programmable logic device (CPLD) 141 that control much of the functionality of the external assembly. In the illustrated configuration, the main processor and/or CPLD 141 control the outputs displayed on the LCD 111, generates the control signals delivered to the vibration device 127 and speaker 125, and receives and processes the signals from buttons 131, 133, 135, microphone 137, and a real-time clock 149. The real-time clock 149 may generate the timing signals that are used with the various components of the system.

The main processor may also manage a data storage device 151, provides redundancy for a digital signal processor 143 (“DSP”), and manage the telemetry circuit 147 and a charge circuit 153 for a power source, such as a battery 155.

While main processor 139 is illustrated as a single processor, the main processor may comprise a plurality of separate microprocessors, application specific integrated circuits (ASIC), or the like. Furthermore, one or more of the microprocessors 139 may include multiple cores for concurrently processing a plurality of data streams.

The CPLD 141 may act as a watchdog to the main processor 139 and the DSP 143 and may flash the LCD 111 and brain state indicators 101 if an error is detected in the DSP 143 or main processor 139. Finally, the CPLD 141 controls the reset lines for the main microprocessor 139 and DSP 143.

A telemetry circuit 147 and antenna may be disposed in the PAD 10 to facilitate one-way or two-way data communication with the implanted device. The telemetry circuit 147 may be an off the shelf circuit or a custom manufactured circuit. Data signals received from the implanted device by the telemetry circuit 147 may thereafter be transmitted to at least one of the DSP 143 and the main processor 139 for further processing.

The DSP 143 and DRAM 145 receive the incoming data stream from the telemetry circuit 147 and/or the incoming data stream from the main processor 139. The brain state algorithms process the data (for example, EEG data) and estimate the subject's brain state, and are preferably executed by the DSP 143 in the PAD. In other embodiments, however, the brain state algorithms may be implemented in the implanted device, and the DSP may be used to generate the communication to the subject based on the data signal from the algorithms in the implanted device.

The main processor 139 is also in communication with the data storage device 151. The data storage device 151 preferably has at least about 7 GB of memory so as to be able to store data from about 8 channels at a sampling rate of between about 200 Hz and about 1000 Hz. With such parameters, it is estimated that the 7 GB of memory will be able to store at least about 1 week of subject data. Of course, as the parameters (e.g., number of channels, sampling rate, etc.) of the data monitoring change, so will the length of recording that may be achieved by the data storage device 151. Furthermore, as memory capacity increases, it is contemplated that the data storage device will be larger (e.g., 10 GB or more, 20 GB or more, 50 GB or more, 100 GB or more, etc.). Examples of some useful types of data storage device include a removable secure digital card or a USB flash key, preferably with a secure data format.

“Subject data” may include one or more of raw analog or digital EEG signals, compressed and/or encrypted EEG signals or other physiological signals, extracted features from the signals, classification outputs from the algorithms, etc. The data storage device 151 can be removed when full and read in card reader 157 associated with the subject's computer and/or the physician's computer. If the data card is full, (1) the subsequent data may overwrite the earliest stored data or (2) the subsequent data may be processed by the DSP 143 to estimate the subject's brain state (but not stored on the data card). While preferred embodiments of the data storage device 151 are removable, other embodiments of the data storage device may comprise a non-removable memory, such as FLASH memory, a hard drive, a microdrive, or other conventional or proprietary memory technology. Data retrieval off of such data storage devices 151 may be carried out through conventional wired or wireless transfer methods.

The power source used by the external assembly may comprise any type of conventional or proprietary power source, such as a non-rechargeable or rechargeable battery 155. If a rechargeable battery is used, the battery is typically a medical grade battery of chemistries such as a lithium polymer (LiPo), lithium ion (Li-Ion), or the like. The rechargeable battery 155 will be used to provide the power to the various components of the external assembly through a power bus (not shown). The main processor 139 may be configured to control the charge circuit 153 that controls recharging of the battery 155.

Further details regarding a seizure advisory system may be found in U.S. patent application Ser. No. 12/020,450, referenced above.

Referring again to FIG. 18, in a preferred embodiment, most or all of the processing of the signals received by the communication unit 208 is done in an external data device 210 that is external to the patient's body. In such embodiments, the communication unit 208 would receive the signals from patient and may or may not pre-process the signals and transmit some or all of the measured signals transcutaneously to an external data device 210, where the prediction of the neurological event and possible therapy determination is made. Advantageously, such embodiments reduce the amount of computational processing power that needs to be implanted in the patient, thus potentially reducing power consumption and increasing battery life. Furthermore, by having the processing external to the patient, the judgment or decision making components of the system may be more easily reprogrammed or custom tailored to the patient without having to reprogram the communication unit 208.

In alternative embodiments, the predictive systems disclosed herein and treatment systems responsive to the predictive systems may be embodied in a device that is implanted in the patient's body, external to the patient's body, or a combination thereof. For example, in one embodiment the predictive system may be stored in and processed by the communication unit 208 that is implanted in the patient's body. A treatment analysis system, in contrast, may be processed in a processor that is embodied in an external data device 210 external to the patient's body. In such embodiments, the patient's propensity for neurological event characterization (or whatever output is generated by the predictive system that is predictive of the onset of the neurological event) is transmitted to the external patient communication assembly, and the external processor performs any remaining processing to generate and display the output from the predictive system and communicate this to the patient. Such embodiments have the benefit of sharing processing power, while reducing the communications demands on the communication unit 208. Furthermore, because the treatment system is external to the patient, updating or reprogramming the treatment system may be carried out more easily.

In other embodiments, the signals 212 may be processed in a variety of ways in the communication unit 208 before transmitting data to the external data device 210 so as to reduce the total amount of data to be transmitted, thereby reducing the power demands of the transmit/receive subsystem 226. Examples include: digitally compressing the signals before transmitting them; selecting only a subset of the measured signals for transmission; selecting a limited segment of time and transmitting signals only from that time segment; extracting salient characteristics of the signals, transmitting data representative of those characteristics rather than the signals themselves, and transmitting only the result of classification. Further processing and analysis of the transmitted data may take place in the external data device 210.

In yet other embodiments, it may be possible to perform some of the prediction in the communication unit 208 and some of the prediction in the external data device 210. For example, one or more characteristics from the one or more signals may be extracted with feature extractors in the communication unit 208. Some or all of the extracted characteristics may be transmitted to the external data device 210 where the characteristics may be classified to predict the onset of a neurological event. If desired, external data device 210 may be customizable to the individual patient. Consequently, the classifier may be adapted to allow for transmission or receipt of only the characteristics from the communication unit 208 that are predictive for that individual patient. Advantageously, by performing feature extraction in the communication unit 208 and classification in an external device at least two benefits may be realized. First, the amount of wireless data transmitted from the communication unit 208 to the external data device 210 is reduced (versus transmitting pre-processed data). Second, classification, which embodies the decision or judgment component, may be easily reprogrammed or custom tailored to the patient without having to reprogram the communication unit 208.

In yet another embodiment, feature extraction may be performed external to the body. Pre-processed signals (e.g., filtered, amplified, converted to digital) may be transcutaneously transmitted from communication unit 208 to the external data device 210 where one or more characteristics are extracted from the one or more signals with feature extractors. Some or all of the extracted characteristics may be transcutaneously transmitted back into the communication unit 208, where a second stage of processing may be performed on the characteristics, such as classifying of the characteristics (and other signals) to characterize the patient's propensity for the onset of a future neurological event. If desired, to improve bandwidth, the classifier may be adapted to allow for transmission or receipt of only the characteristics from the patient communication assembly that are predictive for that individual patient. Advantageously, because feature extractors may be computationally expensive and power hungry, it may be desirable to have the feature extractors external to the body, where it is easier to provide more processing and larger power sources.

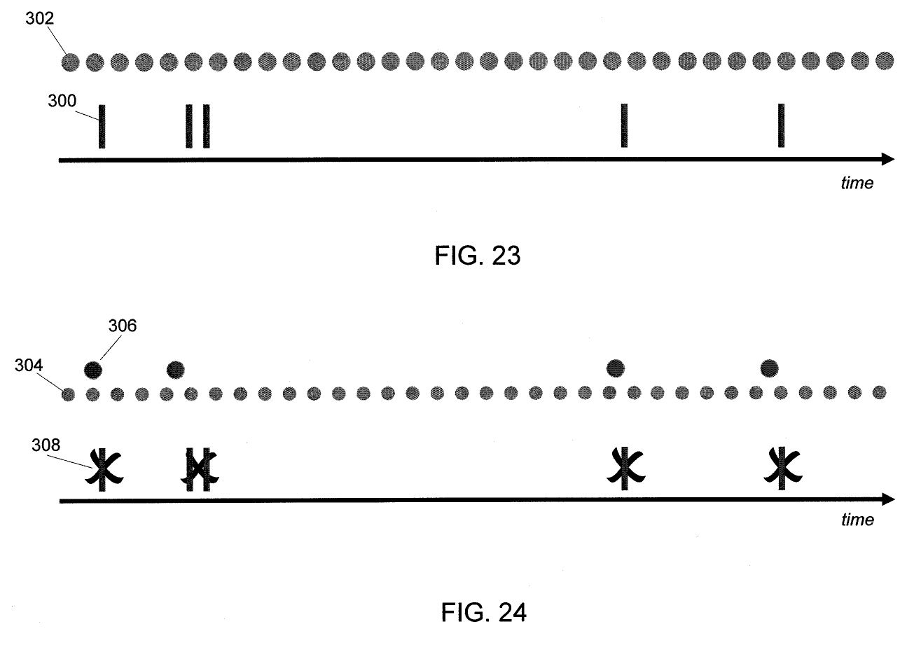

The ability to provide long-term low-power ambulatory measuring of physiological signals and prediction of neurological events can facilitate improved treatment regimens for certain neurological conditions. FIG. 23 depicts the typical course of treatment for a patient with epilepsy. Because the occurrence of neurological events 300 over time has been unpredictable, present medical therapy relies on continuous prophylactic administration of anti-epileptic drugs (“AEDs”). Constant doses 302 of one or more AEDs are administered to a patient at regular time intervals with the objective of maintaining relatively stable levels of the AEDs within the patient. Maximum doses of the AEDs are limited by the side effects of their chronic administration.

Reliable long-term essentially continuously operating neurological event prediction systems would facilitate improved epilepsy treatment. Therapeutic actions, such as, for example, brain stimulation, peripheral nerve stimulation (e.g., vagus nerve stimulation), cranial nerve stimulation (e.g., trigeminal nerve stimulation (“TNS”)), or targeted administration of AEDs, could be directed by output from a neurological event prediction system. One such course of treatment is depicted in FIG. 24. Relatively lower constant doses 304 of one or more AEDs may be administered to a patient at regular time intervals in addition to or as an alternative to the prophylactic administration of the AEDs. Such doses could automatically or manually be delivered with an implanted drug pump or could be administered manually by the patient. Supplementary medication doses 306 may be administered just prior to an imminent neurological event 308. By targeting the supplementary doses 306 at the appropriate times, neurological events may be more effectively controlled and potentially eliminated 308, while reducing side effects attendant with the chronic administration of higher levels of the AEDs.

Prior to enabling the brain state indicators on the user interface 240 of the external data device 210 (FIG. 18), data may be collected from the patient during a training period. The collected data, e.g., an EEG dataset that is indicative of the patient's brain state, may be analyzed to set performance expectations for both the patient and physician and to allow for tailoring of the algorithms to the patient's specific disease state and/or the patient or physician preferences.

The data collected during the training period may be transferred to the physician's workstation 211 or some other central workstation 213 (FIG. 18) where the patient's data may be annotated to identify the patient's seizure activity. Thereafter, the algorithms will be trained on the patient's annotated EEG dataset using the aforementioned statistical methods to set patient specific algorithm parameters. Performance metrics may also be measured for such a patient specific algorithm to set expectations for the physician and patient.

Some examples of data that may be collected and metrics that may be measured include, but is not limited to, number of electrographic seizures, number of clinical seizures, clinical and sub-clinical seizure frequency, average time in a contra-ictal state, average percentage of time in a contra-ictal state (e.g., average percentage of time the patient would have had a green light), negative predictive value, time that elapses after a green light alert ends and a seizure occurs, average time in a pro-ictal state, average percentage of time in a pro-ictal state, percentage of correctly identified electrographic and clinic seizures (sensitivity), positive predictive value, average time interval from when the indication of being in a pro-ictal state would have been enabled and when a seizure actually occurred, time in alert (e.g., contra-ictal or pro-ictal indication), percentage of time in alert, time not in alert (e.g., not contra-ictal or pro-ictal), percentage of time not in alert, or the like.

If the performance metrics indicate that the algorithms are clinically useful for the patient, the algorithms may be uploaded into the system 200 and the brain state indicators may be enabled for use in advising the patient.

After the brain state indicators are enabled, the patient's data will continue to be collected and stored in a memory of the external data device 210 during an assessment period. Such data may subsequently be transferred to the workstations 211, 213 for analysis to assess the continuing performance of the algorithms and allow for further tailoring to the patient or physician preferences. Similar seizure activity data and performance metrics as those measured during the training period may be used to determine if the patient-specific algorithm parameters need to be adjusted. Additionally, other patient seizure activity data, such as un-forewarned seizure activity and number of seizures that occur during a contra-ictal state may be used to assess algorithm performance and patient preferences.

During this assessment period, the physician and patient will have the opportunity to adjust one or more algorithm parameters (e.g., selecting a different operating point) that effect different performance characteristics of the algorithm. However, as can be understood, as one or more performance characteristic of the algorithm is changed, other performance characteristics may be detrimentally affected.

While some patients may prefer certain performance characteristics of their algorithm, such performance characteristics may be wholly unacceptable to another patient. The invention provides systems and methods that allow each particular patient and/or physician to select the operating point that best meets their specific needs.

For example, one subset of patients may prefer an improved sensitivity to determining if they are in a pro-ictal state and don't mind being in alert for a larger percentage of the time, while another subset of patients may prefer a smaller percentage of time in alert and a reduced sensitivity. Furthermore, yet other subsets of patients may place more importance on the contra-ictal indication than the pro-ictal indication and may have specific preferences regarding their desired percentage of time in contra-ictal alert, sensitivity to the contra-ictal state, time period associated with the indication for contra-ictal state (e.g., a green light).

Some examples of complementary aspects of algorithm performance are illustrated in the following Tables. By complementary, it is meant that it is possible to improve a first aspect of performance by sacrificing the second. Furthermore, while the following Tables illustrate only complementary “pairs”, it should be appreciated that the pairs may in fact include a plurality of different performance measures.

For pro-ictal detection, complementary pairs include, but are not limited to:

| PERFORMANCE MEASURE 1 | PERFORMANCE MEASURE 2 |
|---|---|
| Sensitivity (better if larger) | Specificity (better if larger) |
| Sensitivity (better if larger) | Percent time in alert |
| | (better if smaller) |
| Sensitivity (better if larger) | Percent time in false alert |
| | (better if smaller) |

For contra-ictal detection, complementary pairs include, but are not limited to:

| PERFORMANCE MEASURE 1 | PERFORMANCE MEASURE 2 |
|---|---|
| Negative predictive value | percent of time with green light |
| (better if larger) | (better if larger) |
| Specificity (better if larger) | percent of time with green light |
| | (better if larger) |

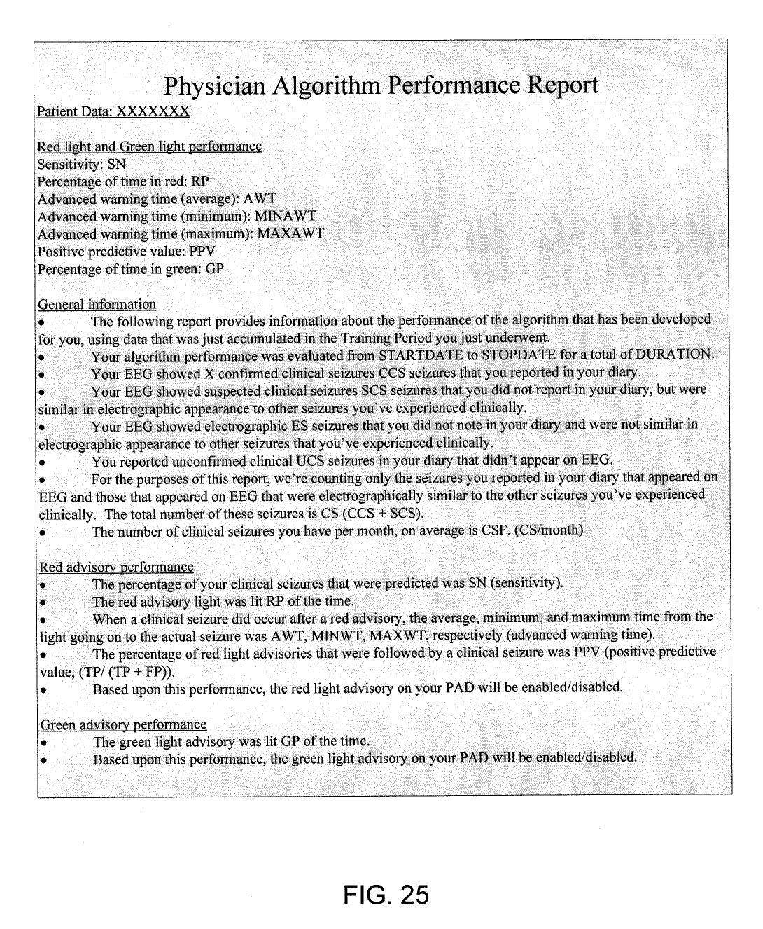

Workstations 211, 213 may be adapted and configured to generate an “algorithm performance report” for the physician and/or patient. The algorithm performance report shown in FIG. 25 may list any number of the aforementioned performance metrics for the patient, and possibly group complementary pairs of the algorithm performance so as to illustrate to the patient the different expected performance metrics for the different operating points.

The following publications are incorporated herein by reference:
Mormann F, Andrzejak R G, Elger C E, Lehnertz K. Seizure prediction: the long and winding road. Brain 2006a.
Mormann F, Elger C E, Lehnertz K. Seizure anticipation: from algorithms to clinical practice. Current Opinion in Neurology 2006b; 19: 187-193.
Schelter B, Winterhalder M, Drentrup H F et al. Seizure prediction: The impact of long prediction horizons. Epilepsy Res 2006.
Winterhalder M, Maiwald T, Voss H U, Aschenbrenner-Scheibe R, Timmer J, Schulze-Bonhage A. The seizure prediction characteristic: a general framework to assess and compare seizure prediction methods. Epilepsy Behav 2003; 4: 318-325.
Wong S, Gardner A B, Krieger A M, Litt B. A Stochastic Framework for Evaluating Seizure Prediction Algorithms Using Hidden Markov Models. J Neurophysiol 2006.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of developing a brain state advisory system comprising:
deriving a brain state advisory algorithm;
applying the brain state advisory algorithm to patient EEG data to identify occurrences of the target patient brain state in the patient EEG data;
determining if a performance measure of the advisory algorithm for the target brain state exceeds the performance measure of a chance predictor for the target brain state; and
if the performance measure of the advisory algorithm for the target brain state exceeds the performance measure of a chance predictor for the target brain state, storing the advisory algorithm in memory of the brain state advisory system.

2. The method of claim 1 wherein the performance measure is a first performance measure, the method further comprising determining an operating point of the chance predictor at which a second performance measure of the chance predictor is substantially the same as the second performance measure of the advisory algorithm prior to determining if the first performance measure of the advisory algorithm exceeds the first performance measure of the chance predictor.

3. The method of claim 2 wherein the first and second performance measures are complementary performance measures.

4. The method of claim 3 wherein one of the first and second performance measures is sensitivity and the other of the first and second performance measures is specificity.

5. The method of claim 3 wherein one of the first and second performance measures is sensitivity and the other of the first and second performance measures is percent time in alert.

6. The method of claim 3 wherein one of the first and second performance measures is negative predictive value and the other of the first and second performance measures is percent time in contra-ictal indication.

7. The method of claim 1 wherein the target brain state is a pro-ictal state.

8. The method of claim 1 wherein the target brain state is a contra-ictal state.

9. The method of claim 1 further comprising generating an alert when the target brain state is identified.

10. A method of monitoring a patient brain state comprising:
obtaining EEG data from the patient;
analyzing the EEG data with a stored brain state advisory algorithm having a performance measure for identification of a target brain state exceeding the performance measure of a chance predictor for the target brain state; and
providing an indication of the target brain state.

11. The method of claim 10 wherein the performance measure is a first performance measure, the analyzing step comprising analyzing the EEG data with a stored brain state advisory algorithm having a first performance measure for identification of a target brain state exceeding the first performance measure of a chance predictor for the target brain state, wherein a second performance measure of the chance predictor for identification of the target brain state is substantially equal to the second performance measure of the stored advisory algorithm for identification of the target brain state.

12. The method of claim 11 wherein the first and second performance measures are complementary performance measures.

13. The method of claim 12 wherein one of the first and second performance measures is sensitivity and the other of the first and second performance measures is specificity.

14. The method of claim 12 wherein one of the first and second performance measures is sensitivity and the other of the first and second performance measures is percent time in alert.

15. The method of claim 12 wherein one of the first and second performance measures is negative predictive value and the other of the first and second performance measures is percent time in contra-ictal indication.

16. The method of claim 10 wherein the target brain state is a pro-ictal state.

17. The method of claim 10 wherein the target brain state is a contra-ictal state.

18. A seizure advisory system comprising:
a seizure advisory algorithm stored in memory, the seizure advisory algorithm having a performance measure for identifying a target brain state greater than the performance measure of a chance predictor for the target brain state;
patient EEG data input;
a microprocessor programmed to apply the algorithm to EEG data from the patient EEG data input to compute patient brain state; and
a patient brain state indicator controlled by the microprocessor to indicate patient brain state.

19. The system of claim 18 wherein the target brain state is a pro-ictal state.

20. The system of claim 18 wherein the target brain state is a contra-ictal state.

21. The system of claim 18 wherein the performance measure is a first performance measure, the seizure advisory algorithm having a first performance measure for identifying the target brain state greater than the first performance measure of a chance predictor for the target brain state, the seizure advisory algorithm having a second performance measure for identifying the target brain state that is substantially equal to the second performance measure of the chance predictor for the target brain state.

22. The system of claim 21 wherein the first and second performance measures are complementary performance measures.

23. The method of claim 22 wherein one of the first and second performance measures is sensitivity and the other of the first and second performance measures is specificity.

24. The method of claim 22 wherein one of the first and second performance measures is sensitivity and the other of the first and second performance measures is percent time in alert.

25. The method of claim 22 wherein one of the first and second performance measures is negative predictive value and the other of the first and second performance measures is percent time in contra-ictal indication.

26. A method of developing a brain state advisory system comprising:
deriving a brain state advisory algorithm, the deriving step comprising
analyzing patient EEG data,
identifying all pro-ictal states within the EEG data, and
generating pro-ictal state alerts;
and
placing the advisory algorithm in memory of the brain state advisory system.

27. The method of claim 26 wherein the patient EEG data comprises EEG data that preceded a seizure by more than 90 minutes.

28. The method of claim 26 wherein the step of identifying all pro-ictal states comprises identifying all pro-ictal states within the patient EEG data without regard to time prior to seizure.

29. The method of claim 26 wherein the deriving step further comprises adjusting sensitivity of the algorithm in identifying pro-ictal states.

30. The method of claim 29 wherein the adjusting step comprises modifying a ratio of number of pro-ictal state alerts generated in the generating step to number of seizures in the EEG data.

31. The method of claim 29 wherein the adjusting step comprises modifying a percentage of time encompassed by pro-ictal alerts generated in the generating step.

32. The method of claim 29 wherein the adjusting step comprises modifying a percentage of time encompassed by pro-ictal alerts generated in the generating step that do not terminate in a seizure.

33. The method of claim 26 wherein identifying all pro-ictal states comprises treating a clustered seizure as a single event.

34. The method of claim 26 wherein generating all pro-ictal state alerts comprises maintaining a pro-ictal alert for a predetermined periodic of time after entering a pro-ictal state.

35. The method of claim 34 wherein the maintaining step comprises maintaining the pro-ictal alert after ceasing to identify a pro-ictal state in the EEG data.

36. The method of claim 35 wherein generating pro-ictal state alerts comprises extending a pro-ictal alert for a second predetermined period of time if a pro-ictal state is again identified after the ceasing step and before the first predetermined period of time has expired.

37. A method of monitoring a patient brain state comprising:
obtaining EEG data from the patient;
analyzing the EEG data with a stored brain state advisory algorithm; and
providing an indication of a pro-ictal brain state for a predetermined period of time after identification of the pro-ictal brain state.

38. The method of claim 37 wherein the providing step comprises continuing the indication of a pro-ictal brain state after the algorithm has ceased to identify a pro-ictal brain state.

39. The method of claim 38 wherein the providing step further comprises extending the indication of a pro-ictal brain state for a second predetermined period of time if the algorithm identifies another pro-ictal state before the first predetermined period of time has expired.

40. A seizure advisory system comprising:
a seizure advisory algorithm stored in memory;
patient EEG data input;
a microprocessor programmed to apply the algorithm to EEG data from the patient EEG data input to identify and indicate patient brain state; and
a patient brain state indicator controlled by the microprocessor to indicate patient brain state for a predetermined period of time after identification of a pro-ictal brain state.

41. The system of claim 40 wherein the microprocessor is programmed to control the patient brain state indicator to indicate patient brain state for a predetermined period of time after identification of a pro-ictal brain state even if the algorithm has ceased to identify a pro-ictal brain state.

42. The system of claim 41 wherein the microprocessor is programmed to control the patient brain state indicator to extend an indication of a pro-ictal brain state for a second pre-determined period of time if the algorithm identifies another pro-ictal brain state before the first predetermined period of time has expired.

43. A method of developing a brain state advisory system comprising:
deriving a brain state advisory algorithm, the deriving step comprising
analyzing patient EEG data,
identifying pro-ictal states within the EEG data, and
generating pro-ictal state alerts;
adjusting a pro-ictal state identification sensitivity of the algorithm; and
storing the advisory algorithm in memory of the brain state advisory system.

44. The method of claim 43 wherein the adjusting step comprises modifying the identifying step.

45. The method of claim 43 wherein the adjusting step comprises modifying the generating step.

46. The method of claim 43 wherein the adjusting step comprises reducing a ratio of number of pro-ictal state alerts generated in the generating step to number of seizures in the EEG data.

47. The method of claim 43 wherein the adjusting step comprises modifying a percentage of time encompassed by pro-ictal alerts generated in the generating step.

48. The method of claim 43 wherein the adjusting step comprises modifying a percentage of time encompassed by pro-ictal alerts generated in the generating step that do not terminate in a seizure.

49. The method of claim 43 wherein the generating step comprises generating alerts each having an alert duration and wherein the adjusting step comprises adjusting a ratio of cumulative alert durations to total time of the EEG data.

50. A method of tailoring a seizure advisory system to a patient, the method comprising:
correlating a first performance measure of the seizure advisory algorithm to a seizure behavior of a subject;
modifying an aspect of the seizure advisory algorithm to improve a second performance measure of the seizure prediction system; and
storing the algorithm in memory in the seizure advisory system.

51. The method of claim 50 wherein the first and second performance measures are complementary performance measures.

52. The method of claim 51 wherein one of the first and second performance measures is sensitivity and the other of the first and second performance measures is specificity.

53. The method of claim 51 wherein one of the first and second performance measures is sensitivity and the other of the first and second performance measures is percent time in alert.

54. The method of claim 51 wherein one of the first and second performance measures is negative predictive value and the other of the first and second performance measures is percent time in contra-ictal indication.

55. The method of claim 50 wherein the seizure behavior comprises a number of seizures in a time interval.

56. The method of claim 50 wherein the seizure advisory algorithm comprises a feature extractor and a classifier.

57. The method of claim 56 wherein modifying an aspect of the seizure advisory algorithm comprises modifying a feature vector analyzed by the seizure prediction system.

58. The method of claim 56 wherein modifying an aspect of the seizure advisory algorithm comprises changing feature extractors or combining the feature extractor with an additional feature extractor.

59. The method of claim 56 wherein modifying an aspect of the seizure advisory algorithm comprises moving or changing a shape of a boundary between classes identified by the classifier.

60. The method of claim 50 wherein modifying an aspect of the seizure advisory algorithm is performed to tailor the seizure advisory system to a particular patient.

61. A method of improving performance of a seizure advisory system, the seizure advisory system comprising a seizure advisory algorithm, the method comprising:
applying the seizure advisory algorithm to a dataset to generate alerts;
extracting information related to alert duration during a time interval of the dataset;
modifying at least one parameter of the seizure advisory algorithm to improve performance of the seizure advisory system; and
placing the seizure advisory algorithm in memory of the seizure advisory system.
